(19) United States
(12) Patent Application Publication
(10) Pub. No.: US 2024/0268792 A1
(43) Pub. Date: Aug. 15, 2024

(54) Systems and Methods for User-Assisted Acquisition of Ultrasound Images

(71) Applicant: Exo Imaging, Inc., Santa Clara, CA (US)

(72) Inventors: Allen Lu, Issaquah, WA (US); Danesh Zonoobi, Edmonton (CA)

(21) Appl. No.: 18/169,745

(22) Filed: Feb. 15, 2023

(51) Int. Cl. A61B 8/08; A61B 8/00

(52) U.S. Cl. CPC A61B 8/5223; A61B 8/463; A61B 8/465; A61B 8/469; A61B 8/54; A61B 8/585

(57) ABSTRACT

A method includes obtaining a first ultrasound image of a living subject and a respective set of control parameters used to acquire the first ultrasound image via an ultrasound device and processing the first ultrasound image of the living subject to obtain one or more attributes of the first ultrasound image. The method includes, in accordance with a determination that the one or more attributes of the first ultrasound image of the living subject meet first criteria, presenting, via a user interface of the computer system, a first set of recommended parameters, different from the respective set of control parameters used to acquire the first ultrasound image, for acquiring a second ultrasound image of the living subject via the ultrasound device; and controlling the ultrasound device to acquire the second ultrasound image of the living subject using the first set of recommended parameters.

### TECHNICAL FIELD

The disclosed implementations relate generally to systems, methods, and devices for utilizing an ultrasound probe.

### BACKGROUND

Ultrasound imaging is an imaging method that uses sound waves to produce images of structures or features within a region probed by the sound waves. In biological or medical applications, ultrasound images can be captured in real-time to show movement of internal organs as well as blood flowing through the blood vessels. The images can provide valuable information for diagnosing and directing treatment for a variety of diseases and conditions.

### SUMMARY

Medical ultrasound is an imaging modality that is based on the reflection of propagating sound waves at the interface between different tissues. Advantages of ultrasound imaging with respect to other imaging modalities may include one or more of: (1) its non-invasive nature, (2) its reduced costs, (3) its portability, and (4) its ability to provide a good temporal resolution, for example on the order of millisecond or better. Point-of-care ultrasound (POCUS) may be used at bedside by healthcare providers as a real-time tool for answering clinical questions (e.g., whether a patient has developmental hip dysplasia). A trained clinician may perform both the task of acquiring and the task of interpreting ultrasound images, without the need for a radiologist to analyze ultrasound images acquired by a highly trained technician. Depending on specifics of the ultrasound examination, there may still be highly specialized training to learn different protocols for acquiring medically relevant images that are high quality images.

In some embodiments, after a patient is positioned in an appropriate way, a clinician positions the ultrasound probe on the body of the patient and manually starts looking for an appropriate (e.g., an optimal) image that allows the clinician to make an accurate diagnosis. Acquiring the proper image may be a time consuming activity that is performed by trial-and-error, and it may require extensive knowledge of human anatomy. For the diagnosis of some conditions, the clinician may need to perform manual measurements on the acquired images. Further, fatigue caused by repetitive tasks in radiology may lead to an increase in the number of diagnostic errors and decreased diagnosis accuracy. There is therefore a need to develop a methodology that allows clinicians to automatically, quickly and reliably, identify and characterize anatomical structures of interest.

Computer aided diagnosis (CAD) systems may help clinicians acquire higher quality images, and automatically analyze and measure relevant characteristics in ultrasound images. The methods, systems, and devices described herein may have one or more advantages, including the ability to provide relevant clinical information to a clinician and provide guidance to clinicians to help the clinician acquire better ultrasound images in less time. For example, in emergency settings, the systems may automatically change operating parameters of an ultrasound probe to obtain acquire ultrasound images that are well-suited to specific anatomical structures.

Portable (e.g., handheld, and/or battery-operated) ultrasound devices are capable of producing high quality images because they contain many transducers (e.g., hundreds or thousands) that can each produce sound waves and receive the echoes for creating an ultrasound image. As disclosed herein, the ultrasound probe (or the computing device) guides the operator by providing guidance on how to position the ultrasound probe so as to obtain a high-quality frame that contains the anatomical structures of interest.

The systems, methods, and devices of this disclosure each have several innovative aspects, the desirable attributes disclosed herein may be derived from one or more of the innovative aspects individually or as a combination, in accordance with some embodiments.

In accordance with some embodiments, a method of acquiring an ultrasound image includes obtaining a first ultrasound image of a living subject and a respective set of control parameters used to acquire the first ultrasound image via an ultrasound device and processing the first ultrasound image of the living subject to obtain one or more attributes of the first ultrasound image. In accordance with a determination that the one or more attributes of the first ultrasound image of the living subject meet first criteria, presenting, via a user interface of the computer system, a first set of recommended parameters, different from the respective set of control parameters used to acquire the first ultrasound image, for acquiring a second ultrasound image of the living subject via the ultrasound device; and controlling the ultrasound device to acquire the second ultrasound image of the living subject using the first set of recommended parameters.

In accordance with some embodiments, an ultrasound probe includes a plurality of transducers and a control unit. The control unit is configured to perform any of the methods disclosed herein.

In accordance with some embodiments, a computer system includes one or more processors and memory. The memory stores instructions that, when executed by the one or more processors, cause the computer system to perform any of the methods disclosed herein.

In accordance with some embodiments of the present disclosure, a non-transitory computer readable storage medium stores computer-executable instructions. The computer-executable instructions, when executed by one or more processors of a computer system, cause the computer system to perform any of the methods disclosed herein.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an ultrasound probe for imaging a patient, in accordance with some embodiments.

FIG. 2 illustrates a block diagram of an ultrasound probe in accordance with some embodiments.

FIG. 3 illustrates a block diagram of a computing device in accordance with some embodiments.

FIG. 4 is a workflow for acquiring ultrasound images, in accordance with some embodiments.

FIG. 5 illustrates an example of automatically rendering scanning assistance, in accordance with some embodiments.

FIG. 6 shows a user interface, in accordance with some embodiments.

FIG. 7 shows an example guidance system, in accordance with some embodiments

FIG. 8A shows a user interface that provides guidance to an operator, in accordance with some embodiments. FIG. 8B shows an example of how changing a tilt of an ultrasound probe affects a plane of the heart that is imaged, in accordance with some embodiments

FIG. 9 shows a measurement user interface, in accordance with some embodiments.

FIG. 10 shows a user interface for acquiring an ultrasound image frame, in accordance with some embodiments.

FIG. 11 shows an example flow process of how measurements of bladder volume are made, in accordance with some embodiments.

FIGS. 12A-12C illustrate a flowchart of a method of acquiring an ultrasound image that includes automatically adjusting parameters of an ultrasound device, in accordance with some embodiments.

Reference will now be made to implementations, examples of which are illustrated in the accompanying drawings. In the following description, numerous specific details are set forth in order to provide a thorough understanding of the present invention. However, it will be apparent to one of ordinary skill in the art that the present invention may be practiced without requiring these specific details.

### DESCRIPTION OF IMPLEMENTATIONS

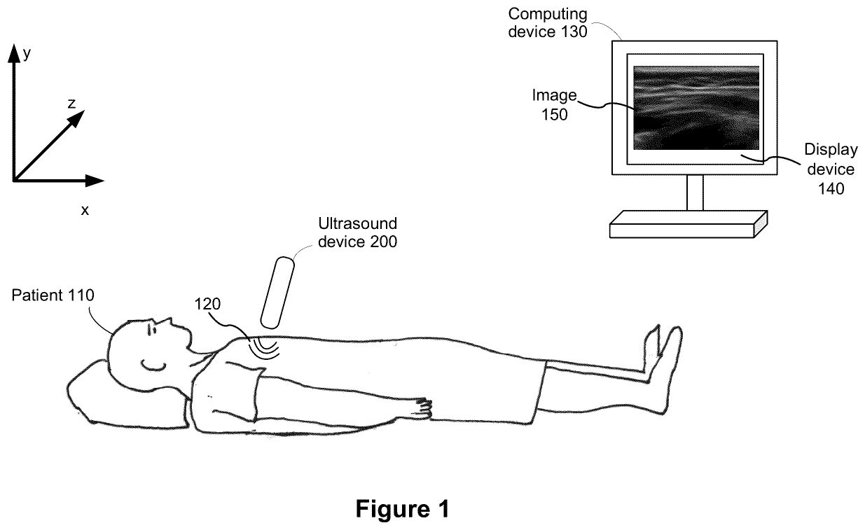

FIG. 1 illustrates an ultrasound system for imaging a patient, in accordance with some embodiments.

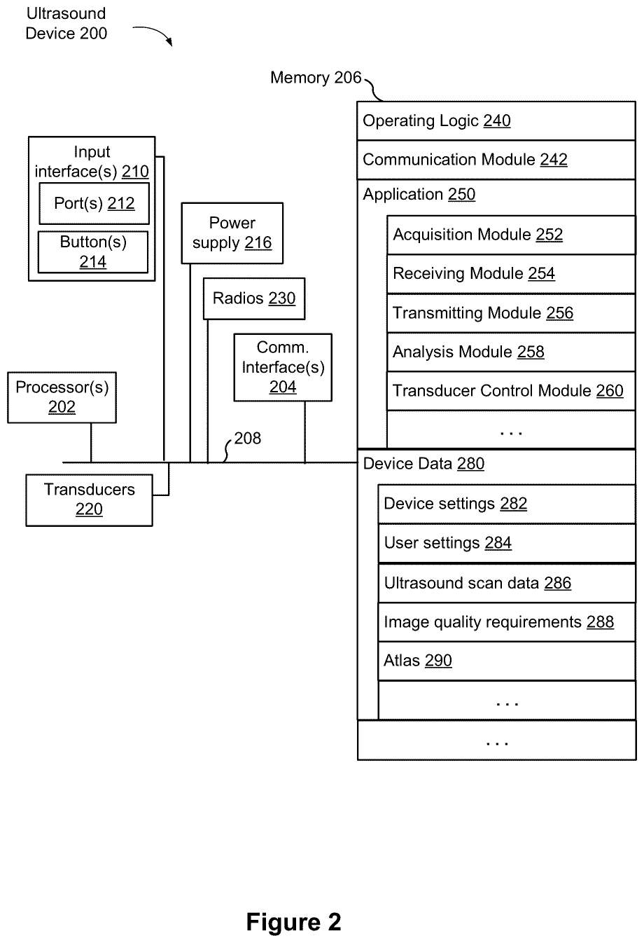

In some embodiments, the ultrasound device 200 is a portable, handheld device. In some embodiments, the ultrasound device 200 includes a probe portion that includes transducers (e.g., transducers 220, FIG. 2). In some embodiments, the transducers are arranged in an array. In some embodiments, the ultrasound device 200 includes an integrated control unit and user interface. In some embodiments, the ultrasound device 200 includes a probe that communicates with a control unit and user interface that is external to the housing of the probe itself. During operation, the ultrasound device 200 (e.g., via the transducers) produces sound waves that are transmitted toward an organ, such as a heart or a lung, of a patient 110. The internal organ, or other object(s) to be imaged, may reflect a portion of the sound waves 120 toward the probe portion of the ultrasound device 200, which are received by the transducers 220. In some embodiments, the ultrasound device 200 transmits the received signals to a computing device 130, which uses the received signals to create an image 150 that is also known as a sonogram. In some embodiments, the computing device 130 includes a display device 140 for displaying ultrasound images, and other input and output devices (e.g., keyboard, touch screen, joystick, touchpad, and/or speakers).

FIG. 2 illustrates a block diagram of an example ultrasound device 200 in accordance with some embodiments.

In some embodiments, the ultrasound device 200 includes one or more processors 202, one or more communication interfaces 204 (e.g., network interface(s)), memory 206, and one or more communication buses 208 for interconnecting these components (sometimes called a chipset).

In some embodiments, the ultrasound device 200 includes one or more input interfaces 210 that facilitate user input. For example, in some embodiments, the input interfaces 210 include port(s) 212 and button(s) 214. In some embodiments, the port(s) can be used for receiving a cable for powering or charging the ultrasound device 200, or for facilitating communication between the ultrasound probe and other devices (e.g., computing device 130, computing device 300, display device 140, printing device, and/or other input output devices and accessories).

In some embodiments, the ultrasound device 200 includes a power supply 216. For example, in some embodiments, the ultrasound device 200 is battery-powered. In some embodiments, the ultrasound device is powered by a continuous AC power supply.

In some embodiments, the ultrasound device 200 includes a probe portion that includes transducers 220, which may also be referred to as transceivers or imagers. In some embodiments, the transducers 220 are based on photo-acoustic or ultrasonic effects. For ultrasound imaging, the transducers 220 transmit ultrasonic waves towards a target (e.g., a target organ, blood vessels, etc.) to be imaged. The transducers 220 receive reflected sound waves (e.g., echoes) that bounce off body tissues. The reflected waves are then converted to electrical signals and/or ultrasound images. In some embodiments, the probe portion of the ultrasound device 200 is separately housed from the computing and control portion of the ultrasound device. In some embodiments, the probe portion of the ultrasound device 200 is integrated in the same housing as the computing and control portion of the ultrasound device 200. In some embodiments, part of the computing and control portion of the ultrasound device is integrated in the same housing as the probe portion, and part of the computing and control portion of the ultrasound device is implemented in a separate housing that is coupled communicatively with the part integrated with the probe portion of the ultrasound device. In some embodiments, the probe portion of the ultrasound device has a respective transducer array that is tailored to a respective scanner type (e.g., linear, convex, endocavitary, phased array, transesophageal, 3D, and/or 4D). In the present disclosure, “ultrasound probe” may refer to the probe portion of an ultrasound device, or an ultrasound device that includes a probe portion.

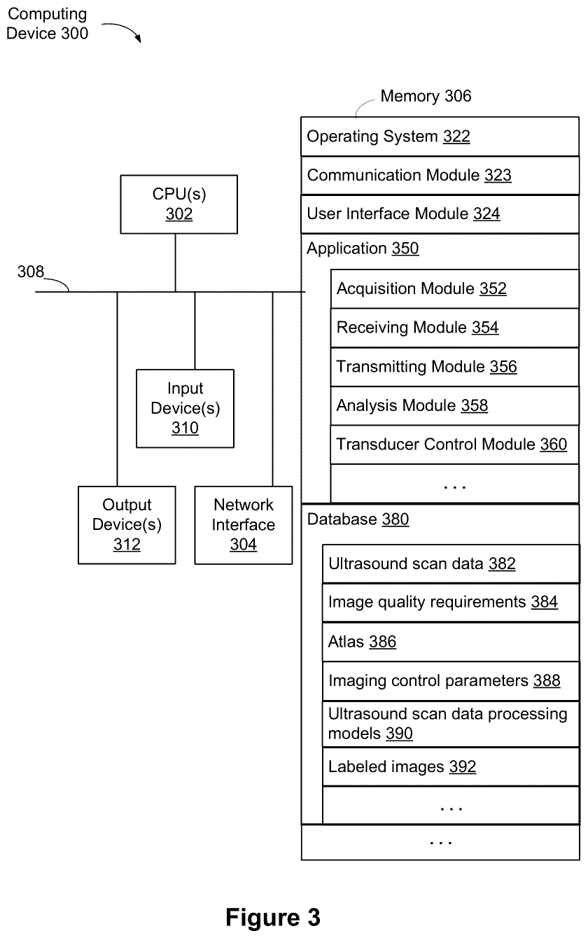

In some embodiments, the ultrasound device 200 includes radios 230. The radios 230 enable one or more communication networks, and allow the ultrasound device 200 to communicate with other devices, such as the computing device 130 in FIG. 1, the display device 140 in FIG. 1, and/or the computing device 300 in FIG. 3. In some implementations, the radios 230 are capable of data communications using any of a variety of custom or standard wireless protocols (e.g., IEEE 802.15.4, Wi-Fi, ZigBee, 6LoWPAN, Thread, Z-Wave, Bluetooth Smart, ISA100.5A, WirelessHART, MiWi, Ultrawide Band (UWB), software defined radio (SDR) etc.) custom or standard wired protocols (e.g., Ethernet, HomePlug, etc.), and/or any other suitable communication protocol, including communication protocols not yet developed as of the filing date of this document.

The memory 206 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 206, optionally, includes one or more storage devices remotely located from one or more processor(s) 202. The memory 206, or alternatively the non-volatile memory within the memory 206, includes a non-transitory computer-readable storage medium. In some implementations, the memory 206, or the non-transitory computer-readable storage medium of the memory 206, stores the following programs, modules, and data structures, or a subset or superset thereof:
operating logic 240 including procedures for handling various basic system services and for performing hardware dependent tasks;
a communication module 242 (e.g., a radio communication module) for connecting to and communicating with other network devices (e.g., a local network, such as a router that provides Internet connectivity, networked storage devices, network routing devices, server systems, computer device 130, computer device 300, and/or other connected devices etc.) coupled to one or more communication networks via the communication interface(s) 204 (e.g., wired or wireless);
application 250 for acquiring ultrasound data (e.g., imaging data) of a patient, and/or for controlling one or more components of the ultrasound device 200 and/or other connected devices (e.g., in accordance with a determination that the ultrasound data meets, or does not meet, certain conditions). In some embodiments, the application 250 includes:
   an acquisition module 252 for acquiring ultrasound data. In some embodiments, the ultrasound data includes imaging data. In some embodiments, the acquisition module 252 activates the transducers 220 (e.g., less than all of the transducers 220, different subset(s) of the transducers 220, all the transducers 220, etc.) according to whether the ultrasound data meets one or more conditions associated with one or more quality requirements;
   a receiving module 254 for receiving ultrasound data;
   a transmitting module 256 for transmitting ultrasound data to other device(s) (e.g., a server system, computer device 130, computer device 300, display device 140, and/or other connected devices etc.);
   an analysis module 258 for analyzing whether the data (e.g., imaging data) acquired by the ultrasound device 200 meets one or more conditions associated with quality requirements for an ultrasound scan. For example, in some embodiments, the one or more conditions include one or more of: a condition that the imaging data includes one or more newly acquired images that meet one or more threshold quality scores, a condition that the imaging data includes one or more newly acquired images that correspond to one or more anatomical planes that match a desired anatomical plane of a target anatomical structure, a condition that the imaging data includes one or more newly acquired images that include one or more landmark/features (or a combination of landmarks/features), a condition that the imaging data includes one or more newly acquired images that include a feature having a particular dimension, a condition that the imaging data supports a prediction that an image meeting one or more requirements would be acquired in the next one or more image frames, a condition that the imaging data supports a prediction that a first change (e.g., an increase by a percentage, or number) in the number of transducer used would support an improvement in the quality score of an image acquired in the next one or more image frames, and/or other analogous conditions; and
   a transducer control module 260 for activating (e.g., adjusting) a number of transducers 220 during portions of an ultrasound scan based on a determination that the ultrasound data meets (or does not meet) one or more quality requirements; and
device data 280 for the ultrasound device 200, including but not limited to:
   device settings 282 for the ultrasound device 200, such as default options and preferred user settings. In some embodiments, the device settings 282 include imaging control parameters. For example, in some embodiments, the imaging control parameters include one or more of: a number of transducers that are activated, a power consumption threshold of the probe, an imaging frame rate, a scan speed, a depth of penetration, and other scan parameters that control the power consumption, heat generation rate, and/or processing load of the probe;
   user settings 284, such as a preferred gain, depth, zoom, and/or focus settings;
   ultrasound scan data 286 (e.g., imaging data) that are acquired (e.g., detected, measured) by the ultrasound device 200 (e.g., via transducers 220);
   image quality requirements data 288. In some embodiments, the image quality requirements data 288 include clinical requirements for determining the quality of an ultrasound image; and
   an atlas 290. In some embodiments, the atlas 290 includes anatomical structures of interest. In some embodiments, the atlas 290 includes three-dimensional representations of the anatomical structure of interest (e.g., hip, heart, lung, and/or other anatomical structures).

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 206 stores a subset of the modules and data structures identified above. Furthermore, the memory 206 may store additional modules or data structures not described above. In some embodiments, a subset of the programs, modules, and/or data stored in the memory 206 are stored on and/or executed by a server system, and/or by an external device (e.g., computing device 130 or computing device 300).

FIG. 3 illustrates a block diagram of a computing device 300 in accordance with some embodiments.

In some embodiments, the computing device 300 is a server or control console that is in communication with the ultrasound device 200. In some embodiments, the computing device 300 is integrated into the same housing as the ultrasound device 200. In some embodiments, the computing device is a smartphone, tablet device, a gaming console, or other portable computing devices.

The computing device 300 includes one or more processors 302 (e.g., processing units of CPU(s)), one or more network interfaces 304, memory 306, and one or more communication buses 308 for interconnecting these components (sometimes called a chipset), in accordance with some implementations.

In some embodiments, the computing device 300 includes one or more input devices 310 that facilitate user input, such as a keyboard, a mouse, a voice-command input unit or microphone, a touch screen display, a touch-sensitive input pad, a gesture capturing camera, or other input buttons or controls. In some embodiments, the computing device 300 uses a microphone and voice recognition or a camera and gesture recognition to supplement or replace the keyboard. In some embodiments, the computing device 300 includes one or more output devices 312 that enable presentation of user interfaces and display content, such as one or more speakers and/or one or more visual displays (e.g., display device 140).

The memory 306 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 306, optionally, includes one or more storage devices remotely located from the one or more processors 302. The memory 306, or alternatively the non-volatile memory within the memory 306, includes a non-transitory computer-readable storage medium. In some implementations, the memory 306, or the non-transitory computer-readable storage medium of the memory 306, stores the following programs, modules, and data structures, or a subset or superset thereof:
an operating system 322 including procedures for handling various basic system services and for performing hardware dependent tasks;
a communication module 323 (e.g., a radio communication module) for connecting to and communicating with other network devices (e.g., a local network, such as a router that provides Internet connectivity, networked storage devices, network routing devices, server systems, computer device 130, ultrasound device 200, and/or other connected devices etc.) coupled to one or more communication networks via the network interface 304 (e.g., wired or wireless);
a user interface module 324 for enabling presentation of information (e.g., a graphical user interface for presenting application(s), widgets, websites and web pages thereof, games, audio and/or video content, text, etc.) either at the computing device 300 or another device;
application 350 for acquiring ultrasound data (e.g., imaging data) from a patient. In some embodiments, the application 350 is used for receiving data (e.g., ultrasound data, imaging data, etc.) acquired via an ultrasound device 200. In some embodiments, the application 350 is used for controlling one or more components of an ultrasound device 200 (e.g., the probe portion, and/or the transducers) and/or other connected devices (e.g., in accordance with a determination that the data meets, or does not meet, certain conditions). In some embodiments, the application 350 includes:
   an acquisition module 352 for acquiring ultrasound data. In some embodiments, the ultrasound data includes imaging data acquired by an ultrasound probe. In some embodiments, the acquisition module 352 activates the transducers 220 (e.g., less than all of the transducers 220, different subset(s) of the transducers 220, all the transducers 220, etc.) according to whether the ultrasound data meets one or more conditions associated with one or more quality requirements. In some embodiments, the acquisition module 352 causes the ultrasound device 200 to activate the transducers 220 (e.g., less than all of the transducers 220, different subset(s) of the transducers 220, all the transducers 220, etc.) according to whether the ultrasound data meets one or more conditions associated with one or more quality requirements;
   a receiving module 354 for receiving ultrasound data. In some embodiments, the ultrasound data includes imaging data acquired by an ultrasound probe;
   a transmitting module 356 for transmitting ultrasound data (e.g., imaging data) to other device(s) (e.g., a server system, computer device 130, display device 140, ultrasound device 200, and/or other connected devices etc.);
   an analysis module 358 for analyzing whether the data (e.g., imaging data, power consumption data, and other data related to the acquisition process) (e.g., received by the ultrasound probe) meets one or more conditions associated with quality requirements for an ultrasound scan. For example, in some embodiments, the one or more conditions include one or more of: a condition that the imaging data includes one or more newly acquired images that meet one or more threshold quality scores, a condition that the imaging data includes one or more newly acquired images that correspond to one or more anatomical planes that match a desired anatomical plane of a target anatomical structure, a condition that the imaging data includes one or more newly acquired images that include one or more landmark/features (or a combination of landmarks/features), a condition that the imaging data includes one or more newly acquired images that include a feature having a particular dimension, a condition that the imaging data supports a prediction that an image meeting one or more requirements would be acquired in the next one or more image frames, a condition that the imaging data supports a prediction that a first change (e.g., an increase by a percentage, or number) in the number of transducer used would support an improvement in the quality score of an image acquired in the next one or more image frames, and/or other analogous conditions; and
   a transducer control module 360 for activating (e.g., adjusting, controlling, and/or otherwise modifying one or more operations of the transducers), or causing the ultrasound device 200 to activate (e.g., via the transducer control module 260), a number of transducers 220 during portions of an ultrasound scan based on a determination that the ultrasound data meets (or does not meet) one or more quality requirements. For example, in some embodiments, the transducer control module 360 activates a first subset of the transducers 220 during the first portion of an ultrasound scan. In some embodiments, the transducer control module 360 activates a second subset of the transducers 220, different from the first subset of the transducers, during a second portion of the scan following the first portion of the scan, when the imaging data corresponding to the first portion of the scan meets (or does not meet) one or more quality requirements. In some embodiments, the transducer control module 360 controls one or more operating modes of the ultrasound device 200. For example, in some embodiments, the ultrasound device 200 is configured to operate in a low-power mode. In the low-power mode, the transducer control module 360 activates only a subset (e.g., 10%, 15%, 20%, etc.) of all the available transducers 220 in the ultrasound device 200. In some embodiments, the ultrasound device 200 is configured to operate in a full-power mode. In the full-power mode, the transducer control module 360 activates all the available transducers 220 to acquire a high-quality image; and
a database 380, including:
   ultrasound scan data 382 (e.g., imaging data) that are acquired (e.g., detected, measured) by one or more ultrasound devices 200;
   image quality requirements data 384. In some embodiments, the image quality requirements data 384 include clinical requirements for determining the quality of an ultrasound image;
   an atlas 386. In some embodiments, the atlas 386 includes anatomical structures of interest. In some embodiments, the atlas 386 includes three-dimensional representations of the anatomical structure of interest (e.g., hip, heart, or lung);
   imaging control parameters 388. For example, in some embodiments, the imaging control parameters include one or more of: a number of transducers that are activated, a power consumption threshold of the probe, an imaging frame rate, a scan speed, a depth of penetration, and other scan parameters that control the power consumption, heat generation rate, and/or processing load of the probe;
   ultrasound scan data processing models 390 for processing ultrasound data. For example, in some embodiments, the ultrasound scan data processing models 390 are trained neural network models that are trained to determine whether an ultrasound image meets quality requirements corresponding to a scan type, or trained to output an anatomical plane corresponding to an anatomical structure of an ultrasound image, or trained to predict, based on a sequence of ultrasound images and their quality scores, whether a subsequent frame to be acquired by an ultrasound probe will contain certain anatomical structures and/or landmarks of interest; and
   labeled images 392 (e.g., a databank of images), including images for training the models that are used for processing new ultrasound data, and/or new images that have been or need to be processed. In some embodiments, the labeled images 392 are images of anatomical structures that have been labeled with their respective identifiers and relative positions.

Each of the above identified elements may be stored in one or more of the memory devices described herein, and corresponds to a set of instructions for performing the functions described above. The above identified modules or programs need not be implemented as separate software programs, procedures, modules or data structures, and thus various subsets of these modules may be combined or otherwise re-arranged in various embodiments. In some embodiments, the memory 306, optionally, stores a subset of the modules and data structures identified above. Furthermore, the memory 306 optionally stores additional modules and data structures not described above. In some embodiments, a subset of the programs, modules, and/or data stored in the memory 306 are stored on and/or executed by the ultrasound device 200.

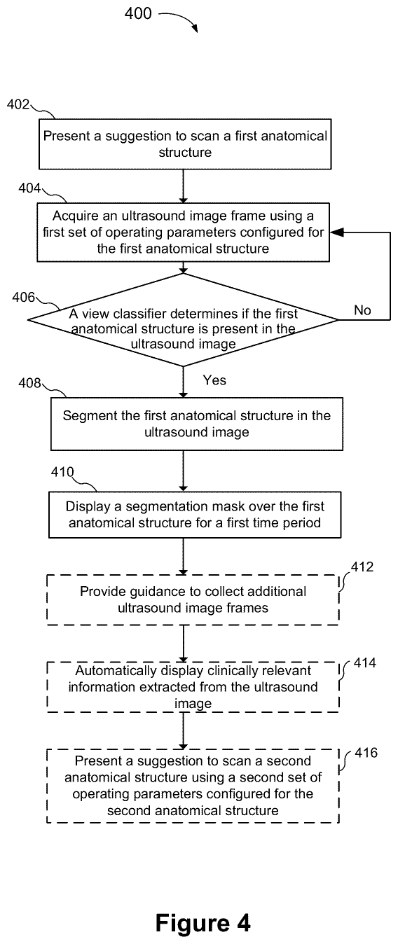

FIG. 4 is a workflow for acquiring ultrasound images, in accordance with some embodiments. In some embodiments, the ultrasound images are medical ultrasound images used for diagnostic purposes. In some embodiments, the workflow 400 is performed by one or more processors (e.g., CPU(s) 302) of a computing device that is communicatively connected with an ultrasound probe. For example, in some embodiments, the computing device is a server or control console (e.g., a server, a standalone computer, a workstation, a smart phone, a tablet device, a medical system) that is in communication with the ultrasound probe. In some embodiments, the computing device is a control unit integrated into the ultrasound probe. In some embodiments, the ultrasound probe is a handheld ultrasound probe or an ultrasound scanning system.

In some embodiments, the workflow 400 includes presenting (402) a suggestion to scan a first anatomical structure. In some embodiments, the suggestion may be an output of a machine learning model that analyzes a real-time ultrasound image frame to determine which anatomical structure is currently being acquired or which anatomical structure has been satisfactorily scanned. Based on that output, the machine learning model provides a suggestion of a next anatomical structure to be acquired. In some embodiments, different anatomical structures may be scanned using corresponding operational parameters (e.g., imaging control parameters) of the ultrasound probe. For example, a frequency, a phase, a duration, power, direction, plane, and/or other operational parameters or configuration of the ultrasound probe may be tailored for respective anatomical structures. In some embodiments, the operational parameters may include a time-varying sequence of frequencies and/or phases, duration, and/or power that is matched to a respective ultrasound collection procedure (e.g., for a particular anatomical structure, or over a particular imaging region or ultrasound probe motion). In some embodiments, operational parameters used to acquire the ultrasound image frames of previously scanned anatomical structure and the subsequent anatomical structure (e.g., the first anatomical structure) are different. In some embodiments, a usage history of the ultrasound probe is used to provide the suggestion.

In response to the detecting a user selection of the suggested first anatomical structure (e.g., by a user input directed to a user interface element associated with beginning a scan of the suggested first anatomical structure), the ultrasound system automatically switches the operational parameters of the ultrasound probe to those corresponding to the first anatomical structure. In some embodiments, there is an automatic change between the operational parameters used to scan the previous anatomical structure and the first anatomical structure. In some embodiments, the operational parameters used to scan the previous anatomical structure are the same as those used to scan the first anatomical structure. The workflow 400 includes acquiring (404) ultrasound image frames of the first anatomical structure using the first set of operating parameters configured for the first anatomical structure.

The ultrasound probe may acquire the images using one or more of its acquisition modalities, including a single (e.g., static) 2D image, an automated sweep yielding a series of (static) images for different 2D planes (referred to herein as a “2D sweep”), a cine clip, which is a real time video capture of scanned anatomy, or a single scan that acquires a 3D volume of images corresponding to a number of different 2D planes (referred to herein as a “3D scan”).

Ultrasound examinations are typically done by placing (e.g., pressing) a portion of an ultrasound device (e.g., an ultrasound probe or scanner) against a surface or inside a cavity of a patient's body, adjacent to the area being studied. An operator (e.g., a clinician) moves the ultrasound device around an area of a patient's body until the operator finds a location and pose of the probe that results in an image of the anatomical structures of interest with sufficiently high quality. In some embodiments, the ultrasound image is an ultrasound image frame that meets one or more quality requirements for making a diagnosis and/or other conditions.

The workflow 400 determines (406) if the currently acquired (e.g., acquired in real-time) ultrasound image includes the first anatomical structure. In some embodiments, a view classifier provides a binary output (e.g., yes or no) regarding whether the first anatomical structure is present in the ultrasound image. In some embodiments, the view classifier is trained using training data that also include anatomical structures other than the first anatomical structure.

In accordance with a determination that the first anatomical structure has not yet been captured, the workflow 400 returns to acquire (404) a new ultrasound image frame until the view classifier determines that the first anatomical structure is present in the acquired ultrasound image.

In accordance with a determination that the first anatomical structure has been captured, the workflow 400 segments (408) the first anatomical structure in the ultrasound image. In some embodiments, the segmentation model runs in a background while ultrasound images are acquired (404) by the ultrasound probe.

In some embodiments, the output of the segmentation step 408 is a segmentation mask that corresponds to a filled outline of the first anatomical image. In some embodiments, the workflow 400 displays (410) the segmentation mask over the first anatomical structure for a first time period before ceasing a display of the segmentation mask. In some embodiments, the first time period is less than three seconds (e.g., less than two seconds, less than one second, less than 0.5 seconds, less than 0.2 seconds). In some embodiments, the display of the segmentation mask provides a visual reminder to an operator that the first anatomical structure has been captured. In some embodiments, a textual label of the first anatomical structure is displayed concurrently with the segmentation mask. In some embodiments, displaying the segmentation mask of the first anatomical structure in a peripheral portion of the real-time ultrasound image frame prompts the operator to reposition the ultrasound probe so that the first anatomical structure is acquired within a central portion of a field of view of the ultrasound probe. In some embodiments, segmentation masks of anatomical structures other than the first anatomical structure are displayed to provide guidance to the operator of a current probing region imaged by the ultrasound probe.

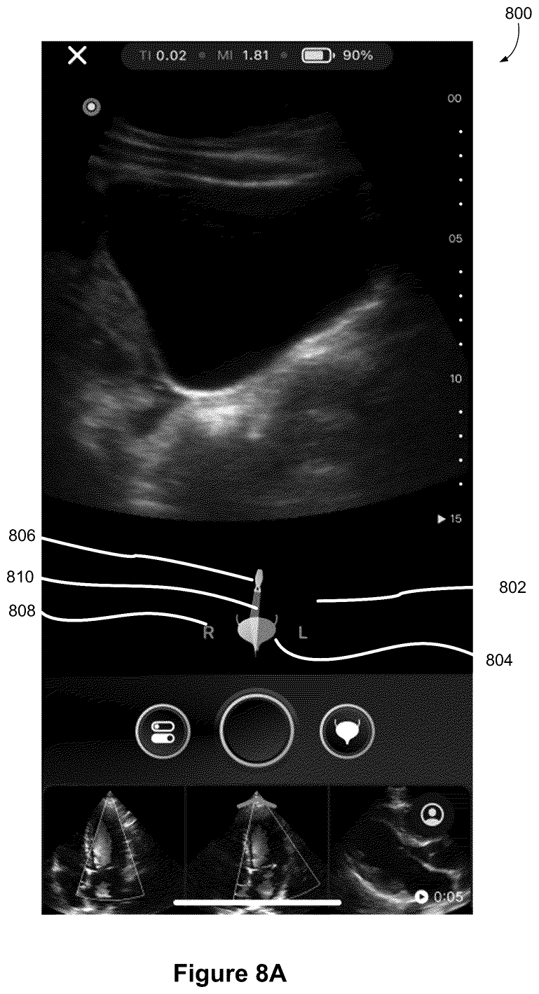

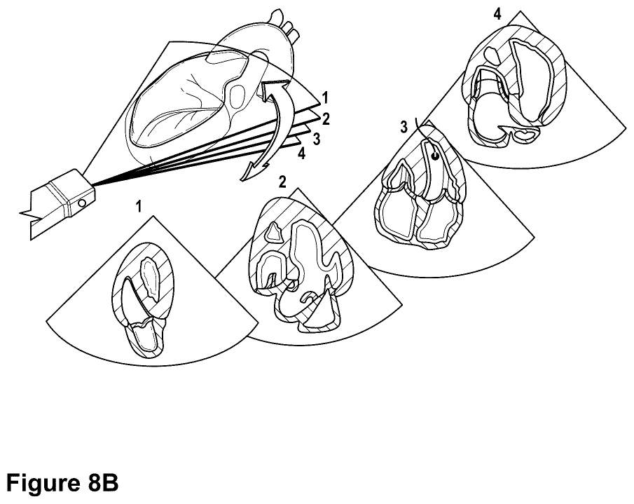

In some embodiments, in accordance with a determination that a view of the first anatomical structure is acquired with sufficient quality, the workflow 400 provides (412) guidance to collect additional ultrasound image frames of the first anatomical structure (e.g., ultrasound image frames of other views of the first anatomical structure), for example, as explained in reference to FIGS. 8A and 8B. A quality of an acquired ultrasound image frame may include, for example, well-defined boundaries (e.g., sufficient contrast) associated with the first anatomical structure.

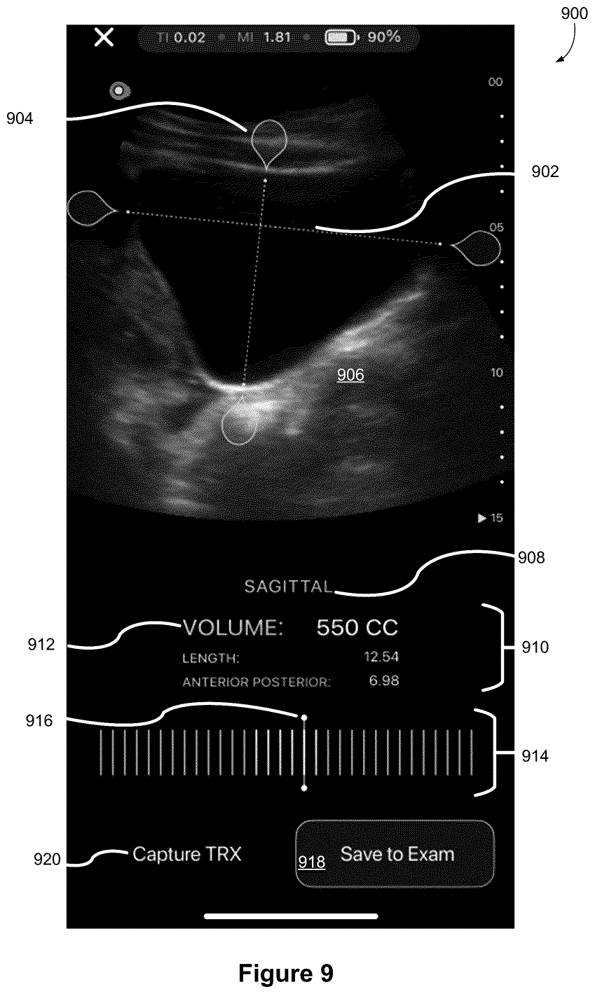

In some embodiments, the workflow 400 includes automatically displaying (414) clinically relevant information extracted from the ultrasound image. For example, as explained in reference to FIG. 9, information about a volume of the first anatomical structure, and one or more linear dimensions associated with the first anatomical structure is displayed.

In some embodiments, the workflow 400 includes determining that ultrasound image acquisition of the first anatomical structure is complete, and presenting (416) a suggestion to scan a second anatomical structure using a second set of operating parameters configured for the second anatomical structure. For example, as described in greater detail below, in some embodiments, an operator will perform a series of scans moving from, e.g., the heart to the bladder. The suggestion in this example may be a suggestion to change from device parameters appropriate for heart scans to device parameters appropriate for bladder scans. In some embodiments, the second set of operating parameters is different from the first set of operating parameters. In some embodiments, the second set of operating parameters is the same as the first set of operating parameters.

In some embodiments, a convolutional neural network for segmentation is used to identify the anatomical regions of interest from the acquired image.

### Training CNN

In some embodiments, the ultrasound image that is acquired in step 404 is used as an input to a trained neural network, such as a convolutional neural network (CNN), which has been trained to determine whether the image complies with all the clinical requirements. In some embodiments, the training involves determining values for a set of weights of the neural network. The output of this network may be one of n classes. When n is 2, the trained neural network may provide a binary output such as “compliant” or “non-compliant.” A compliant image is one that meets the image quality (e.g., clinical) requirements, whereas a non-compliant image is one that does not meet at least one image quality requirement. In some embodiments, the image that is acquired in step 414 is used as an input to a convolutional neural network (CNN) that is trained to output a real number (e.g., from 0 to 1, 0 to 100%, etc.) that indicates a proportion (e.g., a percentage) of the requirements that the image meets. In some embodiments, the neural network is configured (e.g., trained) to provide an indication as to which individual requirements are met and which ones are not.

In some embodiments, the neural network is trained by a training data set that includes a set of p images that have been determined as compliant by a human expert, and a set of q images that have been labeled as non-compliant by a human expert. Each image is then inputted to the convolutional neural network, which is a set of convolutional layers which can optionally be followed by pooling, batch-normalization, dropout, dense, or activation layers. The output of the selected architecture is a vector of length n, where n is the number of classes to be identified. Each entry in the output vector is interpreted as the computed probability of belonging to each of the n classes. The output vector is then compared with a ground truth vector, which contains the actual probability of belonging to each of the n classes (e.g., which may be 100%, in the case of binary labeling). The distance between the output vector and the ground truth vector is then computed using a loss function. Common loss functions are cross-entropy and its regularized versions; however, there are plenty of loss functions that can be used for this process. The loss function is then used to compute an update to the weights. Common optimization methods to compute this update are gradient-based optimization methods, such as gradient descent and its variants. The process of computing the loss and updating the weights is performed iteratively until a predetermined number of iterations is completed, or until a convergence criterion is met. In some embodiments, the neural network is configured to output a real number representing the percentage of requirements that are being currently met by the acquired image. One possible implementation of this approach is to create a set of binary classifiers like the one described above. One binary classifier is trained for each clinical requirement, and the percentage of classifiers with a positive output is then computed.

In some embodiments, the computing device determines an anatomical plane corresponding to an anatomical structure whose image is currently acquired by the ultrasound probe using a trained neural network. In some embodiments, the trained neural network is a trained CNN. In some embodiments, the trained neural network is configured to output a point in 6-dimensional space indicating three positional and three rotational degrees of freedom (e.g., x, y, and z coordinates and pitch, roll, and yaw angles) with respect to the 3D model of the anatomical structure of interest. In some embodiments, the trained neural network can output the angles of the imaging plane in the x-y, y-z, and x-z direction, as well as the distance of the plane to the origin of the 3D model.

In some embodiments, the trained neural network, instead of giving, as an output, a vector representing probabilities, provides values associated with a 6-dimensional vector as an output. In some embodiments, a loss function that is a weighted sum of squared errors or any other loss function suitable for real-valued vectors that are not constrained to be probabilities may also be provided as an output.

In some embodiments, the computing device determines an anatomical plane corresponding to an anatomical structure whose image is currently acquired by the ultrasound probe by partitioning the angle-distance space into discrete classes, and then using a trained neural network that outputs the class of the input image. In some embodiments, the computing device includes (or is communicatively connected with) a bank of images (e.g., a database of images, such as labeled images 392 in the database 380) that has been labeled with their relative positions. The computing device identifies an image in the bank of images that is “closest” to the input image. Here, closest refers to determining the image that minimizes a distance function between the input image and every image in the bank.

In some embodiments, the computing device computes (e.g., measures) a respective distance between an acquired image in a probe-position space (e.g., a six-dimensional space indicating the (x, y, z) position and rotations in the x-, y-, and z-axis with respect to the 3D model of the anatomical structure of interest) and a predicted plane in a probe-position space that would provide a better image, and determines, based on the computation, a sequence of steps that will guide the user to acquire the better image. In some embodiments, the computing device causes the sequence of steps or instructions to be displayed on a display device that is communicatively connected with the ultrasound probe.

In some embodiments, instead of computing a distance between the current image in the probe-position space and a predicted plane, the computing device classifies the current image as one of n possible classes. In some embodiments, changing a tilt of an ultrasound probe can lead to different planes of an organ (e.g., the heart) to be imaged. Because the views of the anatomical planes are well-known in medical literature, a convolutional neural network can be used to learn a classifier that identifies what a current view captured by the image acquired in the step 404 corresponds to.

In some embodiments, the computer system used to implement the workflow 400 may be a local computer system or a cloud-based computer system.

The methods and systems described herein do not assume or require an optimal image, or that an “optimal image” would be the same for every patient who is scanned by the ultrasound probe. The use of an “optimal image” also does not take into account differences in the anatomical positions of organs in different people, or provide any rationale why a big (or a small) deviation is observed in an input image. Thus, in some embodiments, the methods and systems described herein do not include logging deviations between an input image and an optimal image. Instead, the methods and systems segment one or more relevant organs from the obtained images and provide, as an output of the predictor, a set of segmentation masks. The methods and systems described herein identify the respective location(s) of the organs in an image, and are thus robust against differences in the position of the organs across different people.

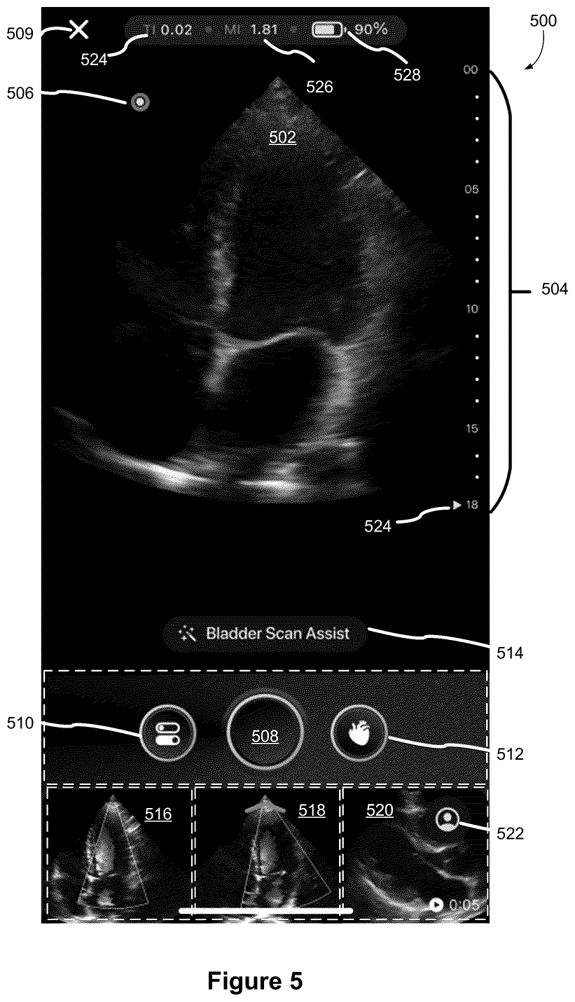

FIG. 5 illustrates an example of automatically rendering scanning assistance, in accordance with some embodiments. FIG. 5 shows a user interface 500. In some embodiments, the user interface 500 is presented on a display of the ultrasound system (e.g., a touch-sensitive screen of a tablet associated with the ultrasound system), and/or the user interface 500 is displayed on an additional screen (a touch-sensitive screen of a cell phone that is communicatively coupled to the ultrasound system, a larger display screen in an examination room, or a display screen in a remote telemedicine clinician's location, and the patient is conducting the ultrasound scan at a location remote from a clinician).

The user interface 500 includes a portion 502 that shows live ultrasound imaging data (e.g., live ultrasound image frames collected in real-time by an ultrasound device 200, live ultrasound image frames displayed at a refresh rate that is configurable by the device 200) collected in real time by an ultrasound imaging probe.

As shown in FIG. 5, an indicator 512 displayed on the user interface 500 provides a visual reminder to an operator of the ultrasound device of the anatomical structure that is being scanned by the ultrasound probe. For example, the indicator 512 depicts a schematic of a heart. In some embodiments, the indicator 512 is displayed in response to a selection by the operator to scan a particular anatomical structure (e.g., a heart, a lung, a hip, a bladder, or a blood vessel).

In some embodiments, the indicator 512 is presented in response to determining the anatomical structure that is currently displayed in the portion 502. For example, in some embodiments, a view classifier model runs in a background as the ultrasound system collects raw ultrasound imaging data. The view classifier may produce a binary output that indicates whether the collected raw ultrasound imaging data corresponds to a particular organ (e.g., a heart). In some embodiments, the view classifier model is trained not only with images of a particular organ, but also includes images of other body anatomies. In some embodiments, in response to the binary output indicating that the raw ultrasound imaging data corresponds to a particular organ (e.g., a heart), an indicator 512 showing a schematic image (e.g., an icon) of the particular organ (e.g., a heart) is shown.

At a lower portion of the user interface 500, previous ultrasound scans collected by the ultrasound system are shown. In some embodiments, an image 516 is collected earlier and was manually saved by the operator of the ultrasound system. In some embodiments, the image 516 is automatically retained by the ultrasound system and displayed to the user. In some embodiments, the image 516 is automatically retained because the image 516 meets a minimum image quality associated with a particular anatomical structure. In some embodiments, the image 516 is representative of a series of scans that were collected or detected by the ultrasound device. In a first nonlimiting example, as shown in FIG. 5, the anatomical structure that is currently scanned corresponds to a heart. In some embodiments, clinical requirements for an echocardiography 4-chamber apical view include: (i) a view of the four chambers (left ventricle, right ventricle, left atrium, and right atrium) of the heart, (ii) the apex of the left ventricle is at the top and center of the sector, while the right ventricle is triangular in shape and smaller in area, (iii) myocardium and mitral leaflets should be visible, and (iv) the walls and septa of each chamber should be visible. In some embodiments, when the ultrasound system (e.g., a localized computer system housed within the ultrasound system, or a cloud-based database communicatively coupled to the ultrasound system) determines that the clinical requirements are met, the corresponding ultrasound image is automatically saved. In some embodiments, each ultrasound image is automatically scored with a quality metric, and the ultrasound system automatically presents the images associated with the highest quality metric scores.

In some embodiments, a second image 518 is also displayed in the lower portion of the user interface 500. Additional information, for example, displayed as coloring overlayed on the images, may be displayed to present pertinent information to the operation. For example, the coloring may show a direction, speed, or velocity of blood flow or the oxygenation level of the blood that is detected by the ultrasound probe. For example, in the image 518 the blue portion may be reflecting oxygenated blood in the right ventricle, and the red portion may show deoxygenated blood flowing into both the left atrium and the right atrium of the patient's heart. A first color (e.g., red) and a second color (e.g., blue) may indicate a blood flow or other information in the ultrasound images or doppler images. For example, blue may denote blood that is going away from a transducer, and red may denote blood that is going towards the transducer. In some embodiments, the images in the lower portion of the user interface 500 are previously acquired ultrasound clips. A third image or video clip 520 may also be displayed in the lower portion of the user interface 500. In some embodiments, a video clip of a scan may be recorded and displayed when a threshold number of frames meeting a quality metric has been obtained within a time period. In some embodiments, the ultrasound system includes logic that causes the ultrasound system to record a video clip if a requisite number of images meeting the quality metric has been obtained. For example, in some embodiments, the ultrasound system has pre-defined logic that determines whether a current video stream if of sufficient quality and the ultrasound system may automatically record the current video stream.

A symbol 522 displayed on the user interface 500 may provide information about the clinician who is conducting the scan. The clinician may have an associated user profile that allows the system to provide more tailored information to the clinician. In some embodiments, the guidance provided by the user is based on one or more fields in the associated user profile. Such fields may include, optionally, a level of training (e.g., radiologist/specialist, primary care doctor, nurse, paramedic, emergency medical technician, etc.); a level of experience (e.g., how many scans of the same type the user has logged); and whether the user has completed specific trainings (e.g., a training for scanning the heart or bladder). For example, if the clinician does not have adequate experience (e.g., a new employee), as indicated in the user profile associated with the symbol 522, the ultrasound device may provide more guidance either visually (e.g., textual guidance), or the ultrasound system may provide audio guidance, or haptic feedback (e.g., vibrations having a changed frequency or changed amplitude to alert the operator that the ultrasound probe is positioned or in an appropriate pose to collect good quality ultrasound images) to the operator as the patient is scanned. In some embodiments, the user interface 500 displays different symbols 522 depending on the one or more fields in the associate user profile (e.g., a different symbol for a radiologist/specialist versus a paramedic).

In contrast, if the clinician is an experienced clinician, fewer guidance instructions or inputs may be provided during the scan processes. In some embodiments, certain image display or collection preferences (e.g., preset settings for contrast, or specific display characteristics for the portion 502) may also be reflected in the user profile associated with the symbol 522.

In some embodiments, while the ultrasound device is collecting a live image of a first anatomical structure (e.g., an organ, such as a heart, or a lung, or a hip), the ultrasound system predicts a next anatomical structure (e.g., a second organ) that is likely to be scanned after the scan of the first anatomical structure is completed. In some embodiments, the user interface 500 provides a prompt 514 of a predicted next anatomical structure to be scanned. For example, the prompt 514 is for a scan assistance associated with a next anatomical structure (e.g., bladder scan assist), as shown in user interface 500. In some embodiments, the prompt 514 includes a pop-up message. In some embodiments, the next anatomical structure to be scanned is based on the user profile (e.g., a user may save a predefined order for an ultrasound evaluation, such as, first scanning the heart, then scanning the bladder, etc.). The system may then assist the user in obtaining a series of scans based on the predefined order, including providing scan assistance and automatically changing presets (e.g., probe parameters), or automatically suggesting changes to presets, as the user moved from one anatomy to the next (e.g., as automatically detected using an image classifier, or based on a user input indicating that one anatomical scan is complete and the user is ready to move to the next).

In some embodiments, the ultrasound system determines the next organ that is likely to be scanned based on prior usage information stored or retrieved by the ultrasound devices. Alternatively, the prediction of the next organ to scan may be based on a contextual setting of the ultrasound device. For example, the ultrasound device may be deployed in an emergency room, or in an ambulance, or in a particular division (e.g., urology department, cardiac department) of a medical institution. In some embodiments, the scan suggestion may also be based on the institution where if it is a specialist clinic associated with a particular organ then the ultrasound system may preferentially suggest to the operator to scan a particular anatomical structure. In some embodiments, a sequence of anatomical structures (e.g., organs) may be on a default scan list to be scanned upon a patient's admission to the emergency room. For example, the heart of the patient may be scanned first, followed by the lung, the kidney, and then the bladder. In some embodiments, each preset scan element in a sequence of scanning workflow may be a state (e.g., a first state associated with a bladder, a second state associated with a lung, or a third state associated with a hip), with probabilities of moving between states based on usage data and characteristics of the operator. Characteristics of the operator may include whether they are an emergency medicine (EM) doctor, hospitalist, or paramedic. In some embodiments, transition probabilities can be based on usage data from other users with the same characteristics (e.g., within or across institutions). In some embodiments, a probability of moving between states is based on previous AI output. For example, in response to an AI algorithm detecting that an ultrasound image captures an inferior vena cava (IVC) having a low collapsibility, the AI algorithm may provide a suggestion to the operator to complete a further ultrasound scan of the heart to evaluate a right atrium of the patient.

In some embodiments, the scan assistance feature is activated once the ultrasound system determines that one or more images (e.g., meeting or exceeding an image quality metric that satisfies minimum requirements for making medical diagnoses) collected for a first anatomical structure, and the ultrasound system is ready to collect raw ultrasound image data of a second anatomical structure (e.g., a second organ such as a bladder). Different anatomical structures may be associated with corresponding operational parameters (e.g., imaging control parameters) for scanning using the ultrasound probe. For example, a frequency, a phase, a duration, power, direction, plane, and/or other operational parameters or configuration of the ultrasound probe may be tailored for respective anatomical structures. In some embodiments, the operational parameters may include a time-varying sequence of frequencies and/or phases, duration, and/or power that is matched to a respective ultrasound collection procedure (e.g., for a particular anatomical structure, or over a particular imaging region or ultrasound probe motion).

For example, a depth or a tissue composition of the anatomical structure that is to be scanned may impact a frequency, phase, or different amplitude of sound waves used to scan the anatomical structure to improve (e.g., optimize) ultrasound signal collection of a particular anatomical structure. In some embodiments, when the scan assist for a particular anatomical structure is invoked, the ultrasound system automatically implements or applies the operational configuration most suitable for scanning the particular anatomical structure without additional input from the operator.

For example, in emergency settings (e.g., in an ambulance, or out in the field, or in an emergency room) when time is critical, an operator may not have time to manually change a scan configuration associated with the ultrasound device to collect ultrasound signals from a particular anatomical structure that is being scanned or is to be scanned. Ultrasound signals collected using mismatched configurations may not be of sufficient quality to render a competent medical diagnosis. Thus, more time may be wasted to repeat the ultrasound scan before a proper diagnosis could be made. In some embodiments, the scan assistance feature is provided as a suggestion system that is more user-friendly and provide suggestions to a user of what the next anatomical structure is to be scanned. A reminder system may be beneficial for an operator under time and stress pressure for the necessary ultrasound images to be collected in order to provide a diagnosis for a patient.

In some embodiments, the suggestion of a next anatomical structure for scanning may be provided based on recognizing the anatomical structure that is currently scanned based on the ultrasound signals that are currently received and/or displayed by the ultrasound system (e.g., in the portion 502) of the user interface 500. In some embodiments, the ultrasound system conducts data analysis of the image that is shown in real time (e.g., in the portion 502) and provides a suggestion of what anatomical structure to scan next.

In some embodiments, the portion 502 is displayed in a top region of the user interface 500 that is easy for an operator (e.g., a clinician, a patient) of the ultrasound device 200 to view during the ultrasound image collection process. In some embodiments, the portion 502 is displayed in a middle region of the user interface 500. In some embodiments, as shown in FIG. 5, the live ultrasound imaging data displayed in the portion 502 has a sector-shaped field of view. In some embodiments, fields of view of other shapes (e.g., rectangular or circular) are displayed in the portion 502, depending on operational configurations of the ultrasound probe and the anatomical feature(s) that are being scanned. In some embodiments, the portion 502 shows live ultrasound imaging data that has undergone some image processing (e.g., data transformation to map the raw ultrasound image into a different coordinate system) and are still updated or refreshed in real time.

On one or more edges of the user interface 500 (e.g., a right edge as shown in FIG. 5, a left edge, a top edge, and/or a bottom edge of the portion 502, or a bottom edge of the user interface 500) is a scale 504 that provides information (e.g., dimensional information) about the ultrasound image that is captured. For example, a marker 524 corresponds a Thermal Index and a marker 526 corresponds to a Mechanical Index, which are indications for a strength of ultrasound energy that is sent into the patient's body. In some embodiments, additional indicators or markers provide information regarding a current plane, depth, or width of an anatomical feature that is being presented in the portion 502. A marker 506 (e.g., a colored dot, or a blue dot) indicates to an operator whether a “mirrored” or horizontally flipped ultrasound image is being presented. In some embodiments, a physical marker (e.g., the physical marker looks like a dot) is placed on (e.g., physically placed on) one side of the transducer as well, and a direction of the physical marker captured on the ultrasound image corresponds to the orientation of the ultrasound image.

As a first nonlimiting example, the clinical requirements for an ultrasound image of a hip to determine the presence of hip dysplasia requires presence of the labrum, ischium, the midportion of the femoral head, flat and horizontal ilium, and absence of motion artifact. As another nonlimiting example, the clinical requirements for an echocardiography 4-chamber apical view are: (i) a view of the four chambers (left ventricle, right ventricle, left atrium, and right atrium) of the heart, (ii) the apex of the left ventricle is at the top and center of the sector, while the right ventricle is triangular in shape and smaller in area, (iii) myocardium and mitral leaflets should be visible, and (iv) the walls and septa of each chamber should be visible. Different sets of requirements for the operating parameters and/or image quality requirements may be implemented based on different scan types, in accordance with various embodiments.

In some embodiments, the user interface 500 also includes various indications about a current state of the ultrasound system. For example, a battery power indicator 528 may show how soon an ultrasound probe may need to be recharged if not directly connected to an AC power source. In some embodiments, the user interface 500 also includes an affordance 510 for surfacing a user interface that displays details about current operational parameters of the ultrasound system (e.g., frequency, contrast, amplitude, phase and/or other parameters). An affordance 508 allows the currently displayed image frame (shown in the portion 502) to be saved (e.g., locally to a computer system housed in the ultrasound system, or to a cloud-based database, for example for computer systems that include mobile devices). A close affordance 509 is also displayed on the user interface 500. A user input directed to the close affordance 509 allows the current scanning session to terminate.

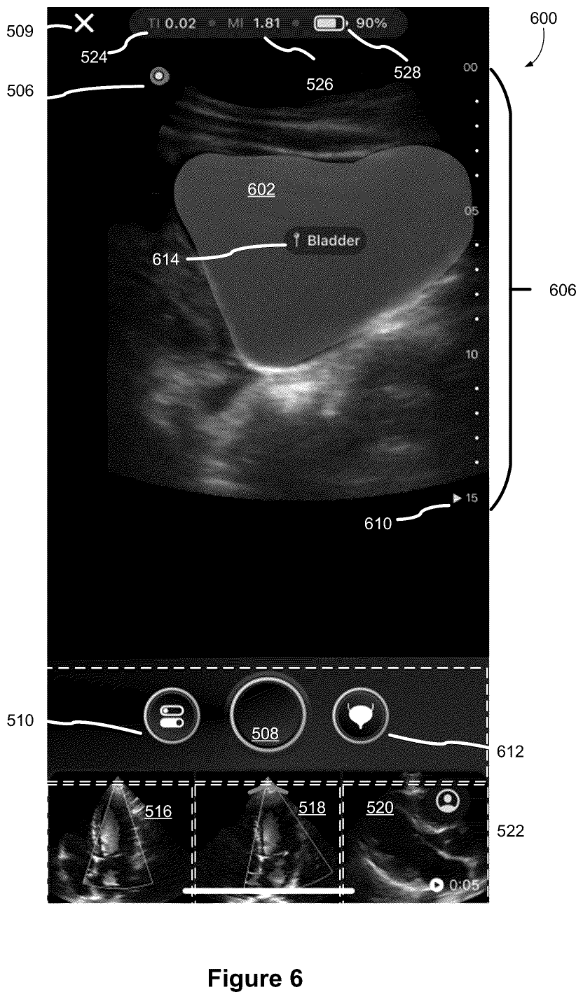

In response to detecting a user input directed at the prompt 514 (e.g., a tap input directed at the prompt 514, a mouse click directed at the prompt 514, a voice-activated selection of the prompt 514, a gaze input directed at the prompt 514 for an ultrasound system that may also include augmented reality or virtual reality capabilities), an updated user interface 600 is displayed as shown in FIG. 6.

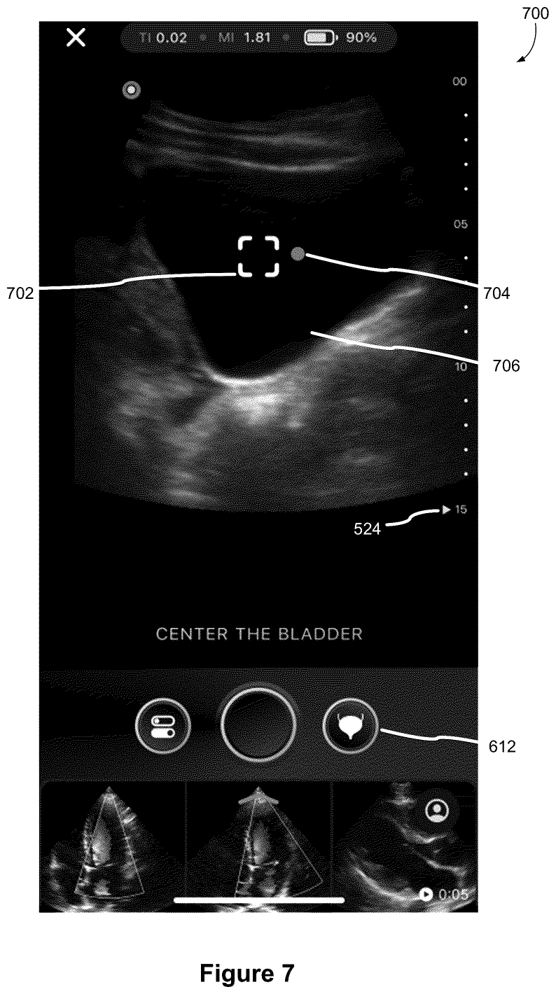

In the updated user interface 600, an indicator 612 is updated to show that a bladder is currently being scanned by the ultrasound system. In some embodiments, a segmentation model is running while the probe is scanning the bladder and helps the user visualize the organ that is being displayed in the live image. In some embodiments, a segmentation mask 602 overlays a live ultrasound image feed (e.g., a live ultrasound image frame). In some embodiments, the segmentation mask 602 showing the bladder (e.g., the segmentation mask is overlaid to cover the anatomical structure having an outline defined by a perimeter of the segmentation mask) is displayed momentarily and the mask is dismissed (e.g., ceases to be displayed) after a period of time (e.g., after 200 millisecond, 500 millisecond, one second, or two seconds). For example, FIG. 7 shows an embodiment in which the segmentation mask is no longer shown overlaid on an anatomical structure 706 with a live ultrasound image frame currently captured by the ultrasound probe. Displaying the segmentation mask of an anatomical structure, and ceasing display of the segmentation mask, helps users who are training, or who are not specialists, quickly locate the anatomy of interest, while allowing the users to view the anatomy of interest unobstructed by the segmentation mask after the segmentation mask is removed. In some embodiments, the operations of displaying the segmentation mask and ceasing to display the segmentation mask are performed in accordance with a mode of the device (e.g., a training or non-specialist mode) and/or one or more fields of a user profile of a user of the device (as described above).

In some embodiments, the ultrasound system first determines whether an anatomical structure is displayed in the portion 502 of the user interface 600. In accordance with a determination that an anatomical structure is presented, the ultrasound system determines what anatomical structure is displayed (e.g., determining that the ultrasound image displayed on the user interface 600 belongs to the bladder class). After the anatomical structure is identified, a predicted segmentation mask is momentarily displayed to overlay the identified anatomical structure.

FIG. 6 shows a user interface, in accordance with some embodiments. The rendering of the segmentation mask 602 in FIG. 6 may, in some embodiments, aid a less experienced operator in the isolating, locating or recognizing that a particular anatomical structure of interest has been at least partially captured or has entered the field of view of the ultrasound system. The display of the segmentation mask provides feedback information to the operator that can speed up the ultrasound data collection process by reducing a chance of the operator repeatedly missing or otherwise failing to capture an image of the anatomical structure that is already within a field of view of the ultrasound probe.

A label 614 may also identify the anatomical structure that is covered by the mask 602. For example, the segmentation mask 602 labeled with the term “bladder” may provide clearer guidance to an operator of what structure is currently shown in the live image portion of the user interface 600.

In some embodiments, the predicted segmentation mask is an output of a segmentation model that includes a multitasking model. The multitasking model includes identification of additional anatomical structures that may not be involved in the original or initial diagnostic goals. In some embodiments, a multitask learning algorithm uses medical expert knowledge to define a mask that contains other relevant anatomical structures. In some embodiments, a multitask learning algorithm generates an output having multiple features (e.g., not an output that provides a single determination or feature). In some embodiments, the medical expert knowledge may be provided, via an atlas of anatomical structures or other medical literature, as information about spatial relationships between various anatomical structures. In some embodiments, the atlas includes a three-dimensional representation of the anatomical structure of interest (e.g., hip, heart, lung, or bladder).

Like the user interface 500, the updated user interface 600 also includes a scale 606 displayed on an edge (e.g., a right edge) of the user interface 600. A marker 610 indicates a dimension (e.g., a width, a height, or a depth) of the anatomical structure displayed on the updated user interface 600.

In some embodiments, even though the ultrasound system is scanning and displaying a second anatomical structure (e.g., a bladder) in real-time, the lower portion of the updated user interface 600 still displays the previously recorded images 516, 518, and 520 of the previously scanned anatomical structure (e.g., the heart). In some embodiments, one or more of the images and video clips displayed on the lower portion of the updated user interface 600 are replaced only after a live scan image is captured, for example by selecting the user interface element 508 to save a copy of the current image frame of the ultrasound image. In some embodiments, a reduced size (e.g., miniaturized) version of the saved image frame is presented on the lower portion of the updated user interface 600.

FIG. 7 shows an example guidance system, in accordance with some embodiments. A first marker 702 displayed in a user interface 700 shown in FIG. 7 indicates a center portion of a field of view of the ultrasound probe. In FIG. 7, the marker 702 shows four (e.g., disjointed) right angles at four corners of a quadrilateral (e.g., a square, or a rectangle). In some embodiments, a guidance system provides an indication of a current center location of the probe to an operator of the probe. In some embodiments, a second marker 704 (e.g., a square marker, a circular colored dot, or a circular red dot) that is visually distinguishable (e.g., easily distinguishable) from a live ultrasound image is rendered on the user interface 700. In some embodiments, as shown in FIG. 7, the live ultrasound image includes the anatomical structure 706 (e.g., a bladder) on which a segmentation mask (e.g., the segmentation mask 602, as shown in FIG. 6) was briefly overlaid.

In some embodiments, the first marker 702 and the second marker 704 are displayed when the ultrasound system determines (e.g., via machine learning models, image recognition techniques or other processing techniques) that a field of view of the ultrasound probe substantially captures an anatomical structure of interest matching a scan type for a current set of preset operational parameters. For example, a current scan type of the ultrasound probe may be a bladder, as shown by the indicator 612. When the ultrasound system analyzes (e.g., via a view classifier) that the currently acquired ultrasound image includes an anatomical structure, a segmentation mask of the identified anatomical structure may be overlaid on the user interface, as shown in FIG. 6.

For applications that include measuring a volume of the bladder, an image frame that captures a center region of the bladder will include a widest dimension of the bladder that would aid in determining the volume of the bladder. In accordance with a determination that the output of the view classifier and/or the segmentation mask matches the scan type (e.g., of a bladder, or a different organ), and that a current view of the ultrasound probe may be further optimized (e.g., to capture a widest width of an anatomical structure by capturing the center portion of the anatomical structure in a central portion of a field of view of the ultrasound probe), the ultrasound system displays the first marker 702 and the second marker 704 on the user interface 700. The first marker 702 and the second marker 704 guide the operator to adjust a position or pose of the ultrasound probe (e.g., a translation of the ultrasound probe to vary an x, y, or z coordinate associated with the probe, a rotation (e.g., fanning) of the ultrasound probe at a particular x, y, z coordinate to change an angle of the ultrasound probe). In some embodiments, when an operator adjusts the position or pose of the ultrasound probe, the movement of the ultrasound probe causes a movement of the first marker 702 so that the second marker 704 is enclosed by the first marker 702 (e.g., a degree of how centered the second marker 704 is within the first marker 702 indicates how well-aligned a particular ultrasound image frame is to a center of the anatomical structure).

In some embodiments, the second marker 704 may denote a center of an anatomical structure that is calculated by a machine learning model, image processing techniques, or other processing techniques, associated with the ultrasound system. In some embodiments the second marker 704 relates to a calculated center of mass of the bladder, which provides an estimate of a center of the bladder. In some embodiment, a machine learning model and/or pre-defined logic may be used to automatically transition the user interface to a next step (e.g., guidance stage) of the scanning process, as illustrated in FIGS. 8A and 8B.

FIG. 8A shows a user interface that provides guidance to an operator, in accordance with some embodiments. In some embodiments, the ultrasound system displays a guidance animation 802 in a user interface 800 to provide guidance to an operator to collect additional ultrasound image data of an anatomical structure. For example, in some embodiments, after the ultrasound probe has been centered based on the guidance provided in FIG. 6, the ultrasound system collects additional ultrasound image frames from different planes of the same anatomical structure. In some embodiments, one or more machine learning models are running in real time as the ultrasound probe is being deployed to scan a patient. The machine learning model may determine that sufficiently high quality images related to a portion of the anatomical structure has been captured and the guidance animation 802 provides a direct visual indication to a user of the remaining portion of the scan that is to be completed.

In some embodiments, the guidance animation 802 is displayed in a portion of the user interface 800 that allows the operator to simultaneously monitor both the real-time ultrasound image frames collected by the ultrasound probe and the guidance animation 802. For example, the animation 802 is displayed in a portion of the user interface 800 that is below the live image frame display portion and above a series of control buttons and the history of the captured in images rendered at the bottom portion of the user interface 800. In some embodiments, the animation 802 is displayed above, on a right portion, or on a left portion of the live image frame display portion. In some embodiments, the animation 802 may be momentarily displayed as being overlaid on (e.g., a portion of) the live image frame display portion. Once one or more target ultrasound image frames are collected, the animation 802 may cease to be displayed.

In some embodiments, for measurements of a bladder, a machine learning model determines from one or more image frames that have been captured (and optionally, additional information from inertial sensors (e.g., an inertia measurement unit)) that a left, a right, a top, or a bottom portion of the bladder has been captured. In some embodiments, the operator makes a fanning motion with the ultrasound probe to capture different planes of the anatomical structure. FIG. 8B shows one example of how changing a tilt (e.g. by making a fanning motion) of an ultrasound probe affects a plane of the heart that is imaged (e.g., different planes of the heart are imaged, depending on the tilt), in accordance with some embodiments. In some embodiments, a convolutional neural network is used to learn a classifier of views of various anatomical planes (e.g., from medical literature) to identify if a real-time ultrasound image frame corresponds to, for example, view 1, view 2, view 3, or view 4 in FIG. 8B.

In some embodiments, while the operator moves the ultrasound probe in a fanning motion, an image of the bladder stays within a field of view of the ultrasound probe (e.g., as captured in the ultrasound image acquired by the probe). In some embodiments, as various 2D image planes are being recorded while the ultrasound probe is being fanned, the ultrasound system determines whether the ultrasound probe has swept through an edge of the bladder, or whether a sufficient number of frames have been recorded to ensure the frame with largest bladder area has been captured. For example, a spheroidal model for the bladder structure is used, and when a diameter of the scanned structure decreases and tapers to a minimum value, the machine learning model (or other processing techniques) determines that an edge of the bladder has been reached and the animation 802 is updated and rendered to show that a portion of the anatomical structure has been scanned. An inertial measurement unit can also be used to measure that both ends of the bladder have been swept through. In some embodiments, the guidance animation includes rendering the corresponding portion of the anatomical structure that has already been scanned and determined to be of sufficient quality for providing diagnostic information, using one or more different visual characteristics (e.g., color, transparency, or shading).

In some embodiments, the guidance animation 802 includes a representation 806 of the ultrasound probe, a representation 804 of the anatomical structure that is being scanned, annotations 808 about orientational or positional information (e.g., left, or right, up, or down) of the anatomical structure, and a scan indication 810 that relates a position of the ultrasound probe to a position of a scanned region of the anatomical structure.

FIG. 9 shows a measurement user interface, in accordance with some embodiments. In some embodiments, once the ultrasound system determines that a scan of the anatomical structure (e.g., an entirety of the anatomical structure) has been completed (e.g., the guidance animation 802 shows that the entire anatomical system has been scanned), the ultrasound system transitions (e.g., automatically transitions, without any user input) into a second measurement mode shown in the user interface 900.

In some embodiments, the user interface 900 displays two orthogonally arranged calipers 902 and 904 that are overlaid on a portion of an image 906. In some embodiments, the image 906 is a saved image frame from a series of measurement image frames. In some embodiments, the image 906 is automatically selected from the series of measurement image frames as the image most likely to provide the most accurate ultrasound image frame for one or more measurements (e.g., a volume measurement based on measuring one or more linear dimensions of an anatomical structure). In some embodiments, the image 906 is a live ultrasound image frame from the ultrasound probe. In some embodiments, instead of displaying, at a lower portion of the user interface, previously captured images and other control affordances (e.g., 510, and 508), various clinically relevant information 910 is displayed based on one or more caliper measurements. For example, a volume 912 of the anatomical structure under study (e.g., bladder volume) is displayed.

In some embodiments, a current view plane of the image 906 is also indicated in the user interface. For example, an indicator 908 (e.g., a textural indicator) states that a sagittal plane is displayed in the image 906. The sagittal plane, in some embodiments, corresponds to a plane in the x-y plane shown in FIG. 1. Some of the clinically relevant information 910 displayed below the image 906 include a length measured by one or more of the calipers and information about a distance between anterior and posterior planes.

A series of vertical lines 914 is also displayed on the user interface 900. In some embodiments, the series of vertical lines 914 represents multiple images that have been captured by the ultrasound system. In some embodiments, vertical lines corresponding to images that are determined to be of sufficient quality are displayed with a different visual characteristic (e.g., color, transparency, or shading) from other image frames (e.g., other saved image frames). For example, vertical lines that are colored blue correspond to image frames that are of sufficient quality. In some embodiments, one or more markers 916 are displayed to indicate which vertical line in the series of vertical lines corresponds to image 906. In some embodiments, the markers indicating which vertical line corresponds to the image 906 include a pair of circles. In some embodiments, a stack of 2D images (e.g., represented by the series of vertical lines 914) is fed to a machine learning model to automatically segment every single frame that is captured to determine the image frame that has the largest bladder. For that image, one or more calipers are overlaid, and a calculated volume calculated based at least in part on the measurements from one of the calipers is displayed to the operator. In some embodiments, the operator is able to select a different ultrasound image frame than the frame automatically selected by machine learning techniques (e.g., due to the image showing a largest dimension of the anatomical structure), by scrolling (e.g. using a swipe input to the left or right) through the series of vertical lines 914.

In some embodiments, a first user element 918, when selected by the operator, allows the image frame 906 to be saved to a medical record for an exam (e.g., associated with a particular patient). In some embodiments, ultrasound image frames for a particular patient acquired and recorded during a particular visit are saved to the exam. In some embodiments, the saved image frames are stored in a computer system housed locally within the ultrasound system. In some embodiments, the saved image frames are stored to a cloud-based database.

In some embodiments, a second user element 920 on the user interface 900, when selected by the operator, allows the operator to capture an image of the anatomical structure (e.g., organ) in a different plane. For example, the different plane is a transverse plane (TRX), corresponding to the y-z plane shown in FIG. 1.

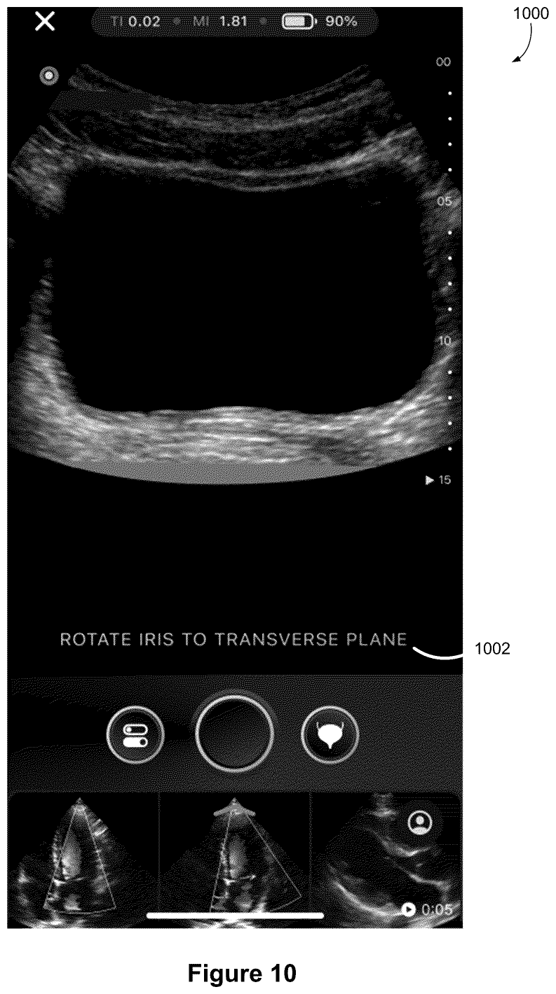

In response to detecting a user input directed at the second user interface element 920 (e.g., a tap input directed at the second user interface element 920, a mouse click directed at the second user interface element 920, a voice-activated selection of the second user interface element 920, a gaze input directed at the second user interface element 920 for an ultrasound system that may also include augmented reality or virtual reality capabilities), a user interface 1000 as shown in FIG. 10 is displayed.

FIG. 10 shows a user interface for acquiring an ultrasound image frame, in accordance with some embodiments. In some embodiments, a prompt 1002 is displayed on the user interface 1000 to guide the operator in acquiring ultrasound image frames of an anatomical structure. In some embodiments, a textual prompt is provided to the operator to rotate the ultrasound probe to capture an ultrasound image plane along a transverse plane. In some embodiments, a live ultrasound image is displayed on the user interface 1000 as the ultrasound probe is used to acquire an ultrasound image. In some embodiments, the operator is able to receive real-time feedback as she repositions the ultrasound probe to acquire ultrasound images in a different plane (e.g., from a sagittal plane to a transverse plane). In some embodiments, once the ultrasound system determines that an ultrasound image from a specific plane (e.g., a transverse plane) has been captured (e.g., by image processing or by analyzing the captured ultrasound image frame using machine learning models), the textual guidance may cease to be displayed.

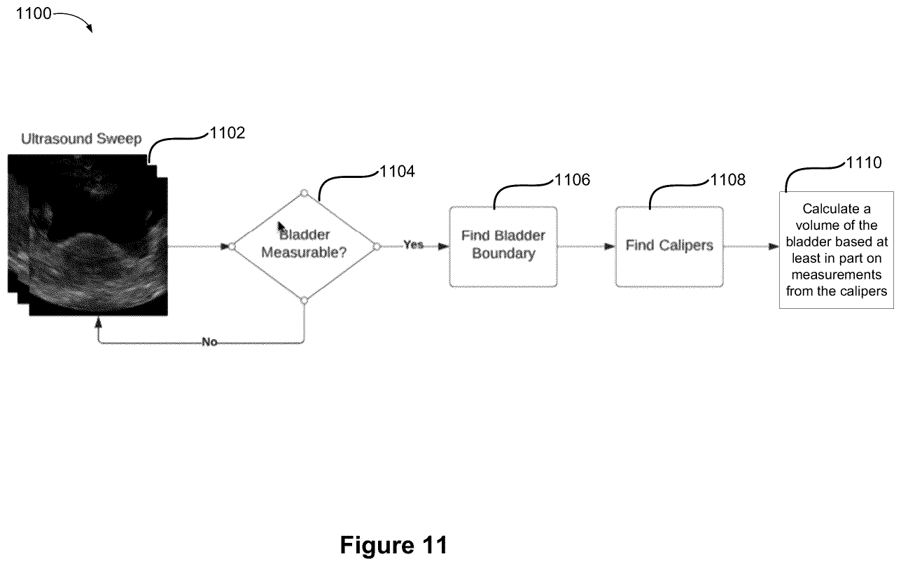

FIG. 11 shows an example flow process of how measurements of bladder volume are made, in accordance with some embodiments. A workflow 1100 starts with a step 1102 for capturing an ultrasound image. The workflow 110 determines (1104) whether the bladder captured in the ultrasound image from the step 1102 corresponds to a bladder that is measurable (e.g., whether the bladder captured in the ultrasound image frame has a well-defined boundary, or whether the entire bladder structure is captured in the ultrasound image frame). In some embodiments, a machine learning model (e.g., a view classifier) determines whether the acquired image contains a measurable bladder.

In accordance with a determination that the bladder is measurable, the workflow 1100 analyzes the ultrasound image frame from the step 1102 to find (1106) a boundary of the bladder within the ultrasound image frame. After the boundary of the bladder in the ultrasound image frame is found, the workflow 1100 selects (1108) one or more calipers for overlaying on the bladder. In some embodiments, positions of the one or more calipers are further fine-tuned by the operator, and a size of a linear dimension of the bladder, and/or a volume of the bladder is presented to the operator.

In some embodiments, a first intersection between a first caliper and the boundary of the bladder is determined, and a second intersection between the first caliper and the boundary of the bladder is determined. A dimension of the bladder is calculated as the distance between the first intersection and the second intersection. The workflow 1100 calculates (1110) a volume of the bladder based at least in part on measurements from the first caliper.

In accordance with a determination that the bladder is not measurable (e.g., no bladder structure is captured in the image, the bladder structure is truncated in the image, the bladder structure is not captured in a view that provides a good contrast for identifying the boundary of the bladder), the workflow 1100 returns to the step 1102 to acquire a new ultrasound image frame, and determines in the step 1104 whether the newly acquired ultrasound image frame includes a bladder that is measurable. The step 1102 is repeated until an ultrasound image frame in which a bladder is measurable is acquired.

In some embodiments, an entire sweep across multiple planes of the organ is captured (e.g., the multiple planes are acquired simultaneously, the multiple planes are acquired sequentially). For each of the captured images, a determination is made as to whether the bladder is measurable (e.g., the step 1104 in the workflow 1100).

In some embodiments, the user interface 500 is presented to the operator during an ongoing ultrasound scan of the bladder and a real-time ultrasound image of the bladder is provided to the operator to better guide the ultrasound scanning process. In some embodiments, the workflow 1100 is started in response to the operator selecting the bladder scan assistance prompt 514 shown in FIG. 5. In some embodiments, the image acquisition step 1102 for obtaining ultrasound images of the bladder includes automatically setting one or more operational parameters of the ultrasound probe (e.g., a frequency, a phase, a duration, power, direction, plane, and/or other operational parameters or configuration) to values that are tailored for the bladder.

In some embodiments, after the step 1110 for calculating a volume of the bladder, a machine learning algorithm running in a background provides suggestions of further anatomical structures to be scanned. In some embodiments, the suggestion or prediction of a next anatomical structure for scanning is provided based on usage data (e.g., historical usage data) of the ultrasound system, and/or the use of a view classifier that runs in the background during an ultrasound scanning process.

In some embodiments, the view classifier model provides a binary output to indicate whether the ultrasound image includes a particular anatomical structure (e.g., a bladder). In some embodiments, the view classifier is trained using images of other body anatomies in addition to the particular anatomical structure (e.g., the view classifier for providing an output about whether the ultrasound image includes a bladder is trained with images of the heart, lung and kidney, in addition to images of the bladder).

In some embodiments, in accordance with a determination (e.g., by the view classifier model) that a bladder is captured in the ultrasound image, a segmentation model running in the background provides a segmentation mask that covers a boundary of the bladder. In some embodiments, the segmentation mask may flash momentarily to serve as a pointer to the operator, while not obscuring the real-time ultrasound images displayed while the ultrasound data is being collected.

In some embodiments, instead of the segmentation mask that identifies a particular anatomical region of interest (e.g., a mask that solely identifies a single anatomical region of interest), the machine learning algorithm that outputs the segmentation mask may use a multitask learning algorithm. In some embodiments, the multitask learning algorithm uses medical expert knowledge to define a second mask that contains other relevant anatomical structures including structures that are not the first anatomical structure or other anatomical regions of interest. In some embodiments, the multitask learning algorithm generates an output having multiple features (e.g., not an output that provides a single determination or feature). In some embodiments, medical expert knowledge is provided by a human expert manually annotating additional anatomical structures in a set of training images and using the manually annotated training images to train the machine learning algorithm to output the second mask. In some embodiments, the medical expert knowledge is provided by a human expert manually annotating training sets that include a collection of new masks that include multiple relevant anatomical structures, in addition to the anatomical feature of interest. In some embodiments, the medical expert knowledge may be provided, via an atlas of anatomical structures or other medical literature, as information about spatial relationships between possible relevant anatomical structures. In some embodiments, the atlas includes a three-dimensional representation of the anatomical structure of interest (e.g., hip, heart, lung, or bladder).

In some embodiments, a machine learning algorithm may include a classifier that identifies a current view associated with the ultrasound image (e.g., the ultrasound image corrected in the step 404), and determines relevant anatomical structures likely to be associated with the current view. In some embodiments, this multitask approach allows the machine learning algorithm to learn a better representation of the ultrasound image that may enhance its performance.

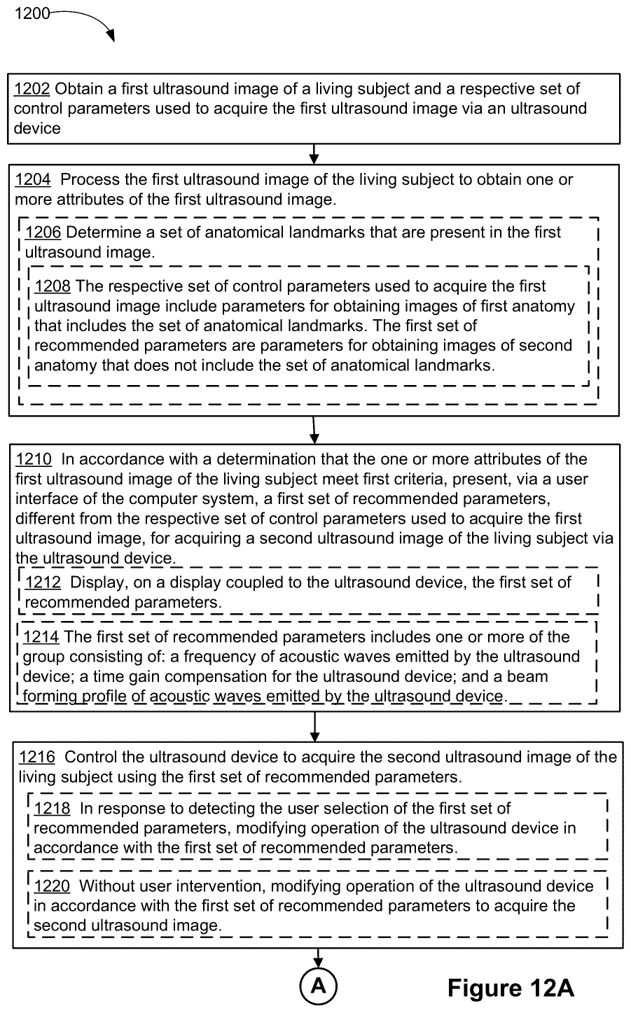

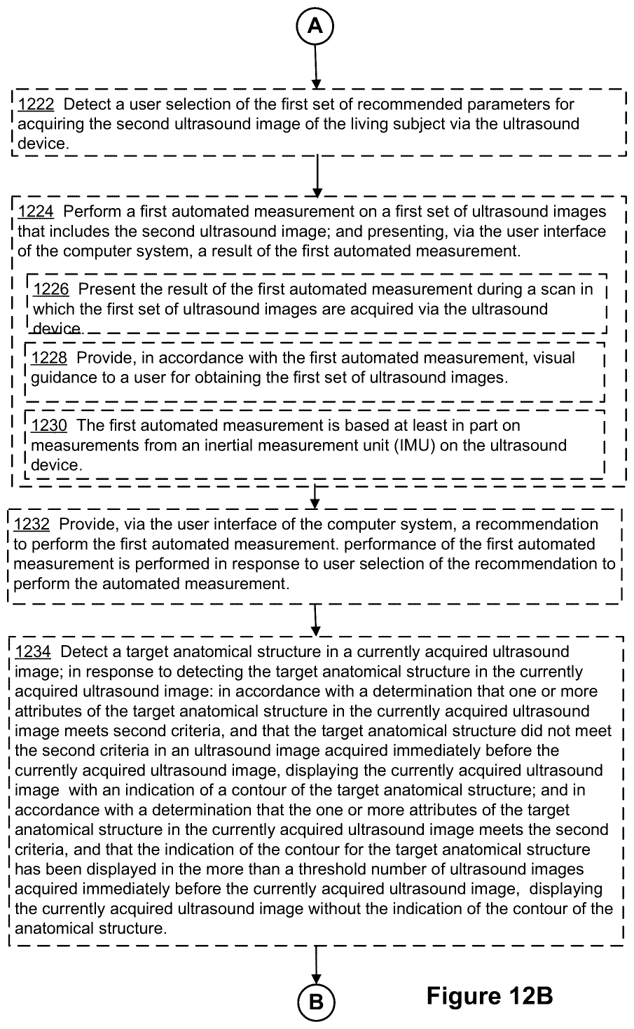

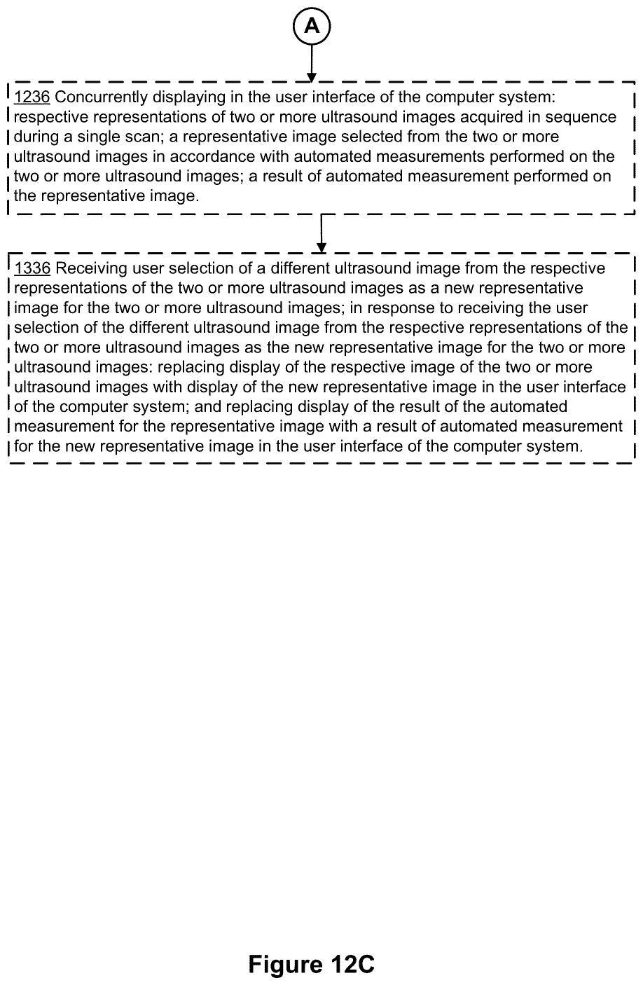

FIGS. 12A-12C illustrate a flowchart diagram of a method 1200 of acquiring an ultrasound image that includes automatically adjusting parameters of an ultrasound device, in accordance with some embodiments. In some embodiments, the ultrasound probe (e.g., ultrasound device 200) is a handheld ultrasound probe, or an ultrasound scanner with an automatic probe. In some embodiments, the method 1300 is performed at a computing device (e.g., computing device 130 or computing device 300) that includes one or more processors (e.g., CPU(s) 302) and memory (e.g., memory 306). For example, in some embodiments, the computing device is a server or control console (e.g., a server, a standalone computer, a workstation, a smart phone, a tablet device, a medical system) that is in communication with a handheld ultrasound probe or ultrasound scanning system. In some embodiments, the computing device is a control unit integrated into a handheld ultrasound probe or ultrasound scanning system.

At a computer system that includes one or more processors and memory, and optionally, an ultrasound device, the computer system obtains (1202) a first ultrasound image of a living subject and a respective set of control parameters used to acquire the first ultrasound image via an ultrasound device. In some embodiments, the living subject is a human. In some embodiments, the living subject is a non-human animal. The computer system processes (1204) the first ultrasound image of the living subject to obtain one or more attributes of the first ultrasound image, for example, by analyzing content of the image. In accordance with a determination that the one or more attributes of the first ultrasound image of the living subject meet first criteria (e.g., the first criteria correspond to a first set of recommended parameters for controlling the ultrasound device), the computer system presents (1210), via a user interface of the computer system (e.g., to a medical provider, and/or other operator of the ultrasound device, such as a physician, nurse, paramedic, or the like), a first set of recommended parameters, different from the respective set of control parameters used to acquire the first ultrasound image (e.g., the respective set of control parameters may be operational parameters) for acquiring a second ultrasound image of the living subject via the ultrasound device.

The computer system controls (1216) the ultrasound device to acquire the second ultrasound image of the living subject using the first set of recommended parameters. In some embodiments, the first criteria correspond to detection of a first set of anatomical landmarks for first anatomy in the first ultrasound image, and the first set of recommended parameters correspond to presets for acquiring images of second anatomy that includes a second set of anatomical landmarks different from the first set of anatomical landmarks.

In some embodiments, the first set of anatomical landmarks is obtained by a geometrical analysis of one or more segmentation masks. For example, in embodiments where the diagnostic target is hip dysplasia, the segmentation mask may be circular (e.g., the femoral head), while the masks for identifying B-lines in lung images may be substantially linear. For example, when the segmentation/identification process provides a circular segmentation mask as an output, the landmark identification process may generate landmarks around a circumference of the circular segmentation mark. In some embodiments, landmarks may be obtained by machine learning and image processing methods. In some embodiments, a statistical model is generated based on landmarks identified from the landmark identification process. For example, landmarks are used as priors in a statistical shape model (SSM) to refine the boundaries of the anatomical regions of interest. A statistical shape model is a geometric model that describes a collection of semantically similar objects and represents an average shape of many three-dimensional objects as well as their variation in shape. In some embodiments, semantically similar objects are objects that have visual (e.g., color, shape, or texture) features that are similar and also similarities in “higher level” information, such as possible relations between the objects. In some embodiments, each shape in a training set of the statistical shape model may be represented by a set of landmark points that is consistent from one shape to the next (e.g., for a statistical shape model involving a hand, the fifth landmark point may always correspond to the tip of the thumb).

For example, an initial circular segmentation mask for the femoral head in diagnosing hip dysplasia may have a diameter of a unit length. Based on the output of the landmark identification process, the circular segmentation mask may be resized to track positions of the landmarks and have a refined circumferential boundary that is larger than a unit length. The refined segmentation masks (or other outputs) may be used to generate a statistical model of the relevant anatomical parts that provides optimized boundaries for organs and/or anatomical structures present in an image. In some embodiments, the landmarks are provided to the statistical shape model to determine if the acquired ultrasound image contains sufficient anatomical information to provide a meaningful clinical decision.

In some embodiments, the computer system detects (1222) a user selection of the first set of recommended parameters for acquiring the second ultrasound image of the living subject via the ultrasound device. In some embodiments, controlling the ultrasound device to acquire the second ultrasound image of the living subject using the first set of recommended parameters includes: in response to detecting the user selection of the first set of recommended parameters, modifying (1218) operation of the ultrasound device in accordance with the first set of recommended parameters.

In some embodiments, the computer system controls the ultrasound device to acquire the second ultrasound image of the living subject using the first set of recommended parameters including: without user intervention, modifying (1220) operation of the ultrasound device in accordance with the first set of recommended parameters to acquire the second ultrasound image.

In some embodiments, the computer system displays (1212), on a display coupled to the ultrasound device, the first set of recommended parameters. In some embodiments, the computer system processes the first ultrasound image of the living subject to obtain one or more attributes of the first ultrasound image includes determining (1206) a set of anatomical landmarks that are present in the first ultrasound image (e.g., using an image segmentation model).

In some embodiments, the respective set of control parameters used to acquire the first ultrasound image include parameters for obtaining images of first anatomy that includes (1208) the set of anatomical landmarks, and the first set of recommended parameters are parameters for obtaining images of second anatomy that does not include the set of anatomical landmarks. In some circumstances, such as in an emergency medical assessment, a medical provider will scan multiple anatomies in succession (e.g., the heart, abdomen, lungs, and bladder). Thus, in some embodiments, the ultrasound device is configured to scan several different anatomies in succession, in a particular order. The computer system determines, using the analysis of the first ultrasound image, which anatomy is currently being scanned and recommends parameters (e.g., presets) for the next anatomy in the particular order. In some embodiments, the computer system also suggests the next anatomy to be scanned (e.g., “Move to bladder and change presets to abdominal presets?”)

In some embodiments, the first set of recommended parameters includes (1214) one or more elements selected from the group consisting of: a frequency of acoustic waves emitted by the ultrasound device; a time gain compensation for the ultrasound device; an imaging depth; a field of view; and a beam forming profile of acoustic waves emitted by the ultrasound device.

In some embodiments, the computer system performs (1224) a first automated measurement on a first set of ultrasound images that includes the second ultrasound image. For example, in some embodiments, after recommending the presets and obtaining a new ultrasound image with the presets, the first automated measurement is performed on the new ultrasound image. In some embodiments, the first set of ultrasound images are obtained from a sweep/scan of a particular anatomy.

In some embodiments, the computer system presents (1226), via the user interface of the computer system, a result of the first automated measurement. In some embodiments, performing the first automated measurement includes determining a physical dimension of an anatomical structure (e.g., a length, area, or volume of the anatomical structure). In some embodiments, the method includes displaying, on the user interface of the computer system, a visual indication of the first automated measurement (e.g., calipers). In some embodiments, the visual indication includes user-modifiable handles, which the user can reposition. In some embodiments, when the user repositions the user-modifiable handles, a corresponding change to the first automated measurement is displayed on the display.

In some embodiments, the first automated measurement is a measurement of bladder volume. In some embodiments, the first automated measurement is a measurement of a vein or artery (e.g., a diameter of the artery or vein (e.g., the inferior vena cava)). In some embodiments, the first automated measurement is a measurement of ejection fraction of a heart of the living subject. In some embodiments, the first automated measurement is a determination of the presence or absence of a diagnostically significant feature of the second ultrasound image. Such diagnostically significant features may include artifacts with known significance (e.g., A-lines and/or B-lines in an ultrasound image of a lung of the living subject). In some embodiments, before performing the first automated measurement of the second ultrasound image, the computer system suggests the first automated measurement to the user. The performance of the first automated measurement, as well as the presentation of the first automated measurement, are then performed in response to a user selection of the suggestion.

In some embodiments, presenting, via the user interface of the computer system, the result of the first automated measurement includes having the computer system presents (1226) the result of the first automated measurement during a scan in which the first set of ultrasound images are acquired via the ultrasound device.

In some embodiments, the computer system provides (1228), in accordance with the first automated measurement, visual guidance to a user for obtaining the first set of ultrasound images. In some embodiments, the first automated measurement is a measurement of a center of an anatomical structure (e.g., the bladder). An indication of the center of the anatomical structure is displayed (e.g., a dot or a cross), on the display, in real-time while the first set of ultrasound images is obtained, allowing the user to aim for the center of the anatomical structure. In some embodiments, the computing system displays a representation of the anatomical structure being scanned (e.g., a cartoon or icon representation of the bladder). The representation of the anatomical structure being scanned fills in as the user captures images of corresponding portions of the anatomical structure, allowing the user to see which portions of the anatomical structure need further scanning. In some embodiments, the first automated measurement is based (1230) at least in part on measurements from an inertial measurement unit (IMU) on the ultrasound device. In some embodiments, the IMU measures an orientation of the device (e.g., three rotational degrees of freedom, such as pitch, roll, and yaw) with respect to a reference frame. In some embodiments, the reference frame is a laboratory reference frame and the living subject is assumed to be in a particular orientation (e.g., lying flat in a supine or prone position) with respect to the laboratory reference frame. In some embodiments, the reference frame is based on analysis of images obtained from the ultrasound device.

In some embodiments, the computer system provides (1232), via the user interface of the computer system, a recommendation to perform the first automated measurement; and (e.g., measure bladder volume). In some embodiments, performance of the first automated measurement is performed in response to user selection of the recommendation to perform the automated measurement. Alternatively, in some circumstances (e.g., depending on the nature of the first automated measurement) performance of the first automated measurement is performed without user intervention.

In some embodiments, during a scan of the living subject using the ultrasound device, the computer system detects a target anatomical structure in a currently acquired ultrasound image; in response to detecting the target anatomical structure in the currently acquired ultrasound image: in accordance with a determination that one or more attributes of the target anatomical structure in the currently acquired ultrasound image meets second criteria, and that the target anatomical structure did not meet the second criteria in an ultrasound image acquired immediately before the currently acquired ultrasound image, display (1234) the currently acquired ultrasound image with an indication of a contour of the target anatomical structure (e.g., acquired ultrasound images are displayed, one at a time, in real-time as the images are obtained).

In some embodiments, in accordance with a determination that the one or more attributes of the target anatomical structure in the currently acquired ultrasound image meets the second criteria, and that the indication of the contour for the target anatomical structure has been displayed in the more than a threshold number of ultrasound images acquired immediately before the currently acquired ultrasound image, displaying the currently acquired ultrasound image without the indication of the contour of the anatomical structure. In some embodiments, the indication of the contour of the anatomical structure is displayed briefly as a guide to the user, and then removed so that the user can better view the anatomical structure. In some embodiments, the indication of the contour is removed after a certain amount of time (e.g., 1 second, 2 seconds). In some embodiments, the indication of the contour is removed after a certain number of images have already been displayed showing the indication of the contour. In some embodiments, the anatomical structure is an anatomical landmark for orienting the user with respect to other anatomies, which may be the subject of diagnosis.

In some embodiments, the computer system concurrently displays (1236) in the user interface of the computer system: respective representations of two or more ultrasound images acquired in sequence during a single scan; a representative image selected from the two or more ultrasound images in accordance with automated measurements performed on the two or more ultrasound images; a result of automated measurement performed on the representative image. In some embodiments, a representation of the respective image is concurrently displayed with the representations of the other images in the second set of ultrasound images. In some embodiments, the representation of the respective image is visually distinguished from the representations of the other images in the second set of ultrasound images (e.g., the representation of the respective image is highlighted or shown in a different color).

In some embodiments, a size of each respective representation of the representations of other images in the second set of ultrasound images is proportional to a result of the automated measurement for the corresponding other image (e.g., the automated measurement is a measurement of the diameter of the inferior vena cava, and each of the representations of the respective image and the other images in the second set of ultrasound images is a line having a length proportional to the measurement of the diameter of the inferior vena cava for the corresponding image).

In some embodiments, each respective representation of the representations of other images indicates whether a result of the second automated measurement is available for the corresponding other image.

In some embodiments, the computer system receives (1336) user selection of a different ultrasound image from the respective representations of the two or more ultrasound images as a new representative image for the two or more ultrasound images; in response to receiving the user selection of the different ultrasound image from the respective representations of the two or more ultrasound images as the new representative image for the two or more ultrasound images: replacing display of the respective image of the two or more ultrasound images with display of the new representative image in the user interface of the computer system; and replacing display of the result of the automated measurement for the representative image with a result of automated measurement for the new representative image in the user interface of the computer system.

In one aspect, an electronic device includes one or more processors; and memory storing instructions that, when executed by the one or more processors, cause the electronic device to perform the method described above in reference to FIGS. 12A-12C.

In another aspect, a non-transitory computer-readable storage medium having stored thereon program code instructions that, when executed by a processor, cause the processor to perform the method described above in reference to FIGS. 12A-12C.

Although some of various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages could be implemented in hardware, firmware, software or any combination thereof.

It will also be understood that, although the terms first, second, etc. are, in some instances, used herein to describe various elements, these elements should not be limited by these terms. These terms are only used to distinguish one element from another. For example, a first transducer could be termed a second transducer, and, similarly, a second transducer could be termed a first transducer, without departing from the scope of the various described implementations. The first sensor and the second sensor are both sensors, but they are not the same type of sensor.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

What is claimed is:

1. A method, comprising:
at a computer system that includes one or more processors and memory:
obtaining a first ultrasound image of a living subject and a respective set of control parameters used to acquire the first ultrasound image via an ultrasound device;
processing the first ultrasound image of the living subject to obtain one or more attributes of the first ultrasound image;
in accordance with a determination that the one or more attributes of the first ultrasound image of the living subject meet first criteria, presenting, via a user interface of the computer system, a first set of recommended parameters, different from the respective set of control parameters used to acquire the first ultrasound image, for acquiring a second ultrasound image of the living subject via the ultrasound device; and
controlling the ultrasound device to acquire the second ultrasound image of the living subject using the first set of recommended parameters.

2. The method of claim 1, further comprising:
detecting a user selection of the first set of recommended parameters for acquiring the second ultrasound image of the living subject via the ultrasound device, wherein controlling the ultrasound device to acquire the second ultrasound image of the living subject using the first set of recommended parameters includes:
in response to detecting the user selection of the first set of recommended parameters, modifying operation of the ultrasound device in accordance with the first set of recommended parameters.

3. The method of claim 1, wherein controlling the ultrasound device to acquire the second ultrasound image of the living subject using the first set of recommended parameters includes:
without user intervention, modifying operation of the ultrasound device in accordance with the first set of recommended parameters to acquire the second ultrasound image.

4. The method of claim 1, wherein presenting, via the user interface of the computer system, the first set of recommended parameters includes, displaying, on a display coupled to the ultrasound device, the first set of recommended parameters.

5. The method of claim 1, wherein processing the first ultrasound image of the living subject to obtain one or more attributes of the first ultrasound image includes determining a set of anatomical landmarks that are present in the first ultrasound image.

6. The method of claim 5, wherein:
the respective set of control parameters used to acquire the first ultrasound image include parameters for obtaining images of first anatomy that includes the set of anatomical landmarks, and
the first set of recommended parameters are parameters for obtaining images of second anatomy that does not include the set of anatomical landmarks.

7. The method of claim 1, wherein the first set of recommended parameters includes one or more elements selected from the group consisting of:
a frequency of acoustic waves emitted by the ultrasound device;
a time gain compensation for the ultrasound device;
an imaging depth;
a field of view; and
a beam forming profile of acoustic waves emitted by the ultrasound device.

8. The method of claim 1, further comprising:
performing a first automated measurement on a first set of ultrasound images that includes the second ultrasound image; and
presenting, via the user interface of the computer system, a result of the first automated measurement.

9. The method of claim 8, further including:
providing, via the user interface of the computer system, a recommendation to perform the first automated measurement; and
wherein performance of the first automated measurement is performed in response to user selection of the recommendation to perform the automated measurement.

10. The method of claim 8, wherein presenting, via the user interface of the computer system, the result of the first automated measurement includes presenting the result of the first automated measurement during a scan in which the first set of ultrasound images are acquired via the ultrasound device.

11. The method of claim 8, including providing, in accordance with the first automated measurement, visual guidance to a user for obtaining the first set of ultrasound images.

12. The method of claim 8, wherein the first automated measurement is based at least in part on measurements from an inertial measurement unit (IMU) on the ultrasound device.

13. The method of claim 1, further comprising: during a scan of the living subject using the ultrasound device:
detecting a target anatomical structure in a currently acquired ultrasound image;
in response to detecting the target anatomical structure in the currently acquired ultrasound image:
in accordance with a determination that one or more attributes of the target anatomical structure in the currently acquired ultrasound image meets second criteria, and that the target anatomical structure did not meet the second criteria in an ultrasound image acquired immediately before the currently acquired ultrasound image, displaying the currently acquired ultrasound image with an indication of a contour of the target anatomical structure; and
in accordance with a determination that the one or more attributes of the target anatomical structure in the currently acquired ultrasound image meets the second criteria, and that the indication of the contour for the target anatomical structure has been displayed in the more than a threshold number of ultrasound images acquired immediately before the currently acquired ultrasound image, displaying the currently acquired ultrasound image without the indication of the contour of the anatomical structure.

14. The method of claim 1, further including:
concurrently displaying in the user interface of the computer system:
respective representations of two or more ultrasound images acquired in sequence during a single scan;
a representative image selected from the two or more ultrasound images in accordance with automated measurements performed on the two or more ultrasound images; and
a result of automated measurement performed on the representative image.

15. The method of claim 14, further including:
receiving user selection of a different ultrasound image from the respective representations of the two or more ultrasound images as a new representative image for the two or more ultrasound images;
in response to receiving the user selection of the different ultrasound image from the respective representations of the two or more ultrasound images as the new representative image for the two or more ultrasound images:
replacing display of the respective image of the two or more ultrasound images with display of the new representative image in the user interface of the computer system; and
replacing display of the result of the automated measurement for the representative image with a result of automated measurement for the new representative image in the user interface of the computer system.

16. A computer system, comprising:
one or more processors; and
memory storing one or more programs, the one or more programs comprising instructions that, when executed by the one or more processors, cause the one or more processors to perform operations comprising:
obtaining a first ultrasound image of a living subject and a respective set of control parameters used to acquire the first ultrasound image via an ultrasound device;
processing the first ultrasound image of the living subject to obtain one or more attributes of the first ultrasound image;
in accordance with a determination that the one or more attributes of the first ultrasound image of the living subject meet first criteria, presenting, via a user interface of the computer system, a first set of recommended parameters, different from the respective set of control parameters used to acquire the first ultrasound image, for acquiring a second ultrasound image of the living subject via the ultrasound device; and
controlling the ultrasound device to acquire the second ultrasound image of the living subject using the first set of recommended parameters.

17. The computer system of claim 16, wherein the memory stores instructions for performing:
detecting a user selection of the first set of recommended parameters for acquiring the second ultrasound image of the living subject via the ultrasound device, wherein controlling the ultrasound device to acquire the second ultrasound image of the living subject using the first set of recommended parameters includes:
in response to detecting the user selection of the first set of recommended parameters, modifying operation of the ultrasound device in accordance with the first set of recommended parameters.

18. A non-transitory computer-readable storage medium, storing a computer program, the computer program, when executed by one or more processors of an electronic device, cause the one or more processors to perform operations comprising:
obtaining a first ultrasound image of a living subject and a respective set of control parameters used to acquire the first ultrasound image via an ultrasound device;
processing the first ultrasound image of the living subject to obtain one or more attributes of the first ultrasound image;
in accordance with a determination that the one or more attributes of the first ultrasound image of the living subject meet first criteria, presenting, via a user interface of the computer system, a first set of recommended parameters, different from the respective set of control parameters used to acquire the first ultrasound image, for acquiring a second ultrasound image of the living subject via the ultrasound device; and
controlling the ultrasound device to acquire the second ultrasound image of the living subject using the first set of recommended parameters.

19. The non-transitory computer-readable storage medium of claim 18, wherein the computer program, when executed by the one or more processors of the electronic device, performs operations including:
detecting a user selection of the first set of recommended parameters for acquiring the second ultrasound image of the living subject via the ultrasound device, wherein controlling the ultrasound device to acquire the second ultrasound image of the living subject using the first set of recommended parameters includes:
in response to detecting the user selection of the first set of recommended parameters, modifying operation of the ultrasound device in accordance with the first set of recommended parameters.
